(19) United States
(12) Patent Application Publication
(10) Pub. No.: US 2025/0166733 A1
(43) Pub. Date: May 22, 2025

(54) DETERMINING STRUCTURAL VARIANTS

(71) Applicant: Illumina, Inc., San Diego, CA (US)

(72) Inventors: Marzieh Eslami Rasekh, Carlsbad, CA (US); Vitor Ferreira Onuchic, San Diego, CA (US); Mitchell A. Bekritsky, Decatur, GA (US)

(21) Appl. No.: 18/949,560

(22) Filed: Nov. 15, 2024

Related U.S. Application Data
(60) Provisional application No. 63/600,492, filed on Nov. 17, 2023.

(51) Int. Cl. G16B 30/10

(52) U.S. Cl. CPC G16B 30/10

(57) ABSTRACT

Described are DNA sequencing systems and methods. Systems and methods may establish a link between read sequences when the sequences are within a threshold distance. The read sequences that are mapped and aligned with high confidence may be used to determine the location of the nearby linked read sequences that would otherwise be difficult to place. The systems and methods may identify structural variants in the polynucleotide by analyzing sequence reads located within a threshold distance to the anchor sequence reads on the flowcell to determine sequence reads linked to the anchor sequence reads.

### CROSS-REFERENCE TO RELATED APPLICATIONS

This application claims priority to U.S. Provisional Application No. 63/600,492, filed Nov. 17, 2023, the content of which is incorporated by reference in its entirety.

### BACKGROUND

### Field

The present disclosure relates to DNA sequencing systems and methods. In particular, this disclosure relates to systems and methods for determining the links between pairs of reads on a sequencing flowcell and using the links to detect and/or determine structural variants.

### Background

Genomic DNA is often too long to be directly sequenced using modern sequencing technologies. Library preparation is a step performed before genome sequencing to facilitate the sequencing process and ensure accurate and efficient analysis of the genomic DNA. Library preparation involves fragmenting the DNA into smaller, manageable pieces. This fragmentation can be achieved through physical or enzymatic methods. Fragmented DNA allows for more efficient sequencing and enables the reconstruction of the original genome during data analysis. Library preparation also involves attaching adapter sequences to the fragmented DNA. Adapters contain specific sequences that are recognized by the sequencing platforms and are necessary for sequencing the DNA fragments. These adapters provide priming sites and identification tags for the sequencing process.

Traditional nucleic acid sequencing methods, and several types of next-generation sequencing methods, use a shotgun approach to sequence large genomic DNA fragments, called template genomic sequences. Specifically, template genomic sequences are first fragmented in solution into smaller pieces that are amenable to next-generation sequencing methods on a flowcell. One of the difficulties of this approach is that by the time the smaller sequence fragments from the template genomic sequences have been read, knowledge of their connectivity and proximity to each other in the original template genomic sequence is lost. The process of ordering the sequence fragments to arrive at the sequence of the original template genomic sequence is generally referred to as “assembly.” Assembly processes can be computationally intensive and time-consuming. In addition, sequence and assembly errors can become a problem depending upon the sequencing methodology used and the quality of genomic DNA samples under evaluation.

### Types of Structural Variants

Structural variants (SVs) are significant genomic alterations that involve changes in the DNA sequence arrangement. These variants encompass various types, each characterized by distinct alterations to the genome's organization. The primary SV types are deletions, duplications, insertions, inversions, and translocations, and they result from different combinations of DNA gains, losses, or rearrangements.

Deletions involve the removal of a segment of DNA, resulting in a missing genomic region. Duplications, on the other hand, lead to the presence of additional copies of a DNA segment, which can result in an increased gene dosage. Insertions entail the insertion of new DNA sequences into the genome, potentially leading to gene disruption or alteration. Inversions denote the reversal of the orientation of a DNA segment, where the sequence order is flipped, but the segment remains within the same chromosome. Translocations, however, involve the movement of genetic material between two different chromosomes or locations, resulting in the fusion of non-adjacent sequences.

These SVs can significantly impact the mapping of short reads to a reference genome during sequencing experiments. The effect of each SV type on read mapping is distinct due to changes in the DNA sequence arrangement.

In the case of deletions, the absence of a segment leads to a reduction in mapped reads spanning the deleted region. This results in a drop in coverage and a noticeable gap in the alignment of reads, leading to a unique pattern in pileup plots. Duplications can lead to excessive coverage in affected regions, causing a higher density of mapped reads. This can sometimes result in confusion in mapping due to the increased number of reads aligning to the duplicated region.

Insertions introduce additional sequences, which can potentially hinder the proper alignment of short reads. The insertion can cause a shift in alignment positions, leading to misalignment or gaps in the alignment. This often results in altered link lengths between paired reads and an irregular distribution of reads around the insertion site.

Inversions disrupt the continuity of the reference sequence, resulting in changes in the orientation of aligned reads within the inverted region. This leads to elongated link lengths and a reversed alignment pattern in pileup plots. The break in alignment pattern at the inversion boundary further complicates accurate mapping.

Translocations create complex alignment scenarios as reads now span multiple chromosomes or locations. This leads to chimeric alignments and can result in abnormal alignment patterns or bridging reads between unexpected genomic locations. This can be particularly challenging for existing mapping process to accurately interpret. In contrast, the disclosed systems and methods may be able to use the complementary spatially links information to detect the presence of SVs.

Structural variants (SVs) encompass a diverse range of genomic alterations, each with unique effects on the arrangement of DNA sequences. Beyond the primary SV types like deletions, duplications, insertions, inversions, and translocations, there are other complex SVs that involve combinations or variations of these alterations. For instance, chromothripsis refers to a catastrophic rearrangement of a chromosome resulting from a single event, leading to a chaotic arrangement of DNA segments. Another example is tandem duplications, where segments are duplicated and tandemly arranged, potentially leading to gene amplification.

The impact of these structural variants on mapping short reads to a reference genome can be profound and varies based on the nature of the alteration. The effects on mapping process and resulting alignment patterns are influenced by the changes in sequence organization and continuity caused by SVs.

Deletions result in the removal of genomic segments, leading to a decreased number of aligned reads spanning the deleted region. Consequently, the coverage drops in the deleted region, creating a noticeable gap in the alignment pattern. This gap reflects a decrease in aligned reads, which is evident in pileup plots and coverage plots.

Duplications introduce extra copies of DNA sequences, leading to an increase in the number of aligned reads over the duplicated region. This can result in elevated coverage and confusion in mapping due to the higher density of reads aligning to the duplicated area. Additionally, the non-uniform distribution of reads within the duplicated segment can impact the accuracy of mapping process.

Insertions introduce new sequences into the genome, causing shifts in alignment positions for reads that span the insertion site. This leads to misalignment or gaps in the alignment pattern, resulting in an irregular distribution of aligned reads around the insertion point.

Inversions disrupt the linear orientation of DNA segments, leading to reversed alignment patterns within the inverted region. This effect elongates the link lengths between paired reads, indicating the rearrangement in the genome. The break in alignment continuity at the inversion boundary further complicates accurate mapping. Translocations involve the movement of genetic material between chromosomes or locations. This generates chimeric alignments, where reads span multiple chromosomes or regions. Existing mapping systems struggle to interpret these bridging reads, often leading to misalignment and inaccurate read placements. Inversions and translocations are also more likely to occur between regions with highly similar DNA sequence (e.g., segmental duplications), again making it difficult to even detect the presence of the rearrangement.

Complex SVs like chromothripsis and tandem duplications introduce highly disordered genomic arrangements, leading to extremely fragmented and chaotic alignment patterns. The resulting alignment disruptions make it challenging for conventional mapping processes to accurately interpret the sequencing data.

### SUMMARY

This disclosure describes a method of detecting structural variants that involve mutations in a polynucleotide in comparison to a reference genome. This is achieved by analyzing reads that are in close proximity on the flow cell surface. Unlike traditional methods which rely on analyzing the entirety of sequencing data, this approach analyzes sequence reads located within clusters on a flowcell. The systems or methods may determine the location of clusters of “anchor” sequence reads, where an anchor sequence read is a read which has a well-known position in the genome. Such positions are generally not mutated or repeated in the genome and thus are more readily determined with a relatively high level of confidence. The systems or methods may then determine the position of other reads on the flowcell and calculate a threshold distance from particular reads to anchor sequence reads on the flowcell. This provides a targeted approach to determine links between sequence reads that may be within a structural variant or span the relevant area of a structural variant. By linking the unknown sequence read to an anchor read, the systems or methods can determine the actual position of the unknown sequence read within an individual's genome with high confidence. This approach reduces the volume of data that needs to be parsed, making the detection of structural variants more efficient and faster. The method also improves the accuracy and specificity of variant identification by focusing on a specific, predefined area around anchor reads. This approach is better than broader methods which might miss nuanced structural variants or require a large volume of sequencing data.

Furthermore, the disclosure provides for determining sequence reads that are specifically linked to the anchor sequence reads from sequencing methods that provide sequence reads with a probability of being located near each other on the flowcell that is correlated with a distance between the fragments. By establishing this linkage, one can deduce the placement of a putative structural variants with a spatial relationship with the anchor sequence. Systems and methods are also provided for detecting a structural variant using complementary sequencing information, where the complementary information includes the spatial location of the sequence and the links between sequences. A baseline metric for the distribution of links for a low probability of structural variants may be used to determine whether variations in the number or distribution of spatially linked sequences is significant and could indicate the presence of a structural variant. Additionally, by more effectively identifying structural variants, the system can filter for or filter out reads where candidate structural variants were detected, thus improving efficiency of the overall system.

Aspects of the disclosure relate to a system for identifying structural variants in a polynucleotide, including: a memory; and at least one processor configured to perform a method, the method including: obtaining sequence reads from a flowcell including fragments of a polynucleotide, wherein a probability of the fragments of the polynucleotide being located near each other on the flowcell is correlated with a distance between the fragments of the polynucleotide in the polynucleotide; determining anchor sequence reads flanking putative structural variants in the polynucleotide; and identifying structural variants in the polynucleotide by analyzing sequence reads located within a threshold distance to the anchor sequence reads on the flowcell to determine sequence reads linked to the anchor sequence reads. Some methods may not rely on a strict threshold and may use, for example, quality scores for links between reads on the flowcell.

Some aspects relate to a method for identifying structural variants in a polynucleotide, including: obtaining sequence reads from a flowcell including fragments of a polynucleotide, wherein a probability of the fragments of the polynucleotide being located near each other on the flowcell is correlated with a distance between the fragments of the polynucleotide in the polynucleotide; determining anchor sequence reads flanking putative structural variants in the polynucleotide; and identifying structural variants in the polynucleotide by analyzing sequence reads located within a threshold distance to the anchor sequence reads on the flowcell to determine sequence reads linked to the anchor sequence reads.

Some aspects relate to a method of identifying genomic variants in a polynucleotide including: providing genomic data including polynucleotide sequence reads and coordinates of the polynucleotide sequences from the polynucleotide on a sequencing substrate; aligning the polynucleotide sequence reads to a reference genome; selecting aligned polynucleotide sequence reads which are within a predetermined distance from one another on the sequencing substrate; determining a genomic distance between the alignments on the reference genome of the aligned polynucleotide sequence reads with the selected polynucleotide sequence reads; and identifying a polynucleotide as having a candidate genomic variant, when the aligned polynucleotide sequence reads are within the predetermined distance and have a genomic distance above a calculated value.

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear. While the disclosure has been illustrated and described in detail in the drawings and foregoing description, such illustration and description are to be considered illustrative or exemplary and not restrictive. The disclosure is not limited to the disclosed embodiments. Variations to the disclosed embodiments can be understood and effected by those skilled in the art in practicing the claimed disclosure, from a study of the drawings, the disclosure and the appended claims.

FIG. 1 schematically illustrates a non-limiting example of a solid support which can perform embodiments of the disclosed sequencing technology.

FIG. 2 shows a flowchart of an example method for determining the links between pairs of reads on a sequencing flowcell and using the links to detect structural variants.

FIG. 3 shows a colocation heatmap that shows the relationships between linked read pairs in the Factor VIII gene.

FIG. 4 displays a colocation heatmap representing the relationships among linked read pairs across different regions of a gene, believed to be a version of the Factor VIII gene.

FIG. 5 illustrates an example of process of identifying subpairs linked to breakpoints in genomic data.

FIG. 6 presents a multi-layered alignment plot designed to illustrate the alignment of reads to the Human GRCh38/hg38 reference genome within a specific genomic region.

FIG. 7 illustrates an example pipeline designed for structural variant detection in genomic data.

FIG. 8 is a block diagram of an exemplary computing system that may be used in connection with an illustrative sequencing system.

### DETAILED DESCRIPTION

All patents, applications, published applications and other publications referred to herein are incorporated herein by reference to the referenced material and in their entireties. If a term or phrase is used herein in a way that is contrary to or otherwise inconsistent with a definition set forth in the patents, applications, published applications and other publications that are herein incorporated by reference, the use herein prevails over the definition that is incorporated herein by reference.

Embodiments relate to systems and methods for determining the presence of a structural variant in a polynucleotide. Because structural variants may involve repeated sequences, or deletions or duplications of DNA, embodiments link sequence reads which cannot be mapped to a reference genome with a threshold mapping quality (MAPQ) to anchor sequences which have a strong mapping quality to a nearby genomic region. This linkage between the sequence read with a low MAPQ and related anchor sequences is found by using the physical location of each sequence read and anchor sequence on the flowcell to help properly assign the sequence read to its correct location on an original polynucleotide from the genome.

In some next generation sequencing (NGS) systems, fragments of long DNA, such as genomic DNA, from a biological source are sheared to create shorter fragments which can be sequenced in a single read. The shearing process can create shorter fragments which land on the flowcell and the spatial location of each fragment may be related to the original nucleic acid molecule from which the fragment was derived. For example, fragments which come from the same nucleic acid molecule land closer together on the flowcell as compared to fragments which come from different original nucleic acid molecules. Accordingly, if two clusters of reads on a flowcell are close together spatially and also close together on the genome, the clusters generated on the flowcell are more likely to have come from the same original nucleic acid molecule.

However, it should be realized that unrelated fragments may also bind to the flowcell near one another, which leads to an uncertainty in the probability that adjacent clusters originate from the same molecule. A number of factors could affect the probability that unrelated clusters would be generated in a similar area, and these factors may change based on a variety of experimental conditions. Embodiments of the invention provide a statistical method for calculating the probability that two reads are linked, such that on a flowcell the two reads were derived from the same nucleic acid molecule.

Some embodiments provide for establishing the quality of a link between two or more read pairs on a flowcell. The “link” as discussed herein is the probability that two pairs of reads on a sequencing flowcell are derived from the same original nucleic acid molecule. In some embodiments, the link between two pairs of reads on a sequencing flowcell does not require a quantifiable metric to determine the quality of the link between two reads.

Embodiments of the invention relate to systems and methods for sequencing target nucleic acids by fragmenting the target nucleic acid and distributing the fragments onto a flowcell. As the fragments are distributed along the flowcell, they bind capture primers and are then used to create clusters by well-known technologies, such as those provided by Illumina Inc. (San Diego, CA). As described above, fragments which were derived from the same template genomic sequence are more likely to bind to the flowcell in spatially nearby positions as compared to fragments that are from different template genomic sequences, particularly when the fragmentation is performed directly on the flowcell using immobilized transposome complexes on the surface of the flowcell. This spatial information can be used to help guide assembly and variant calling of the original template genomic sequence, as will be described in more detail below.

For example, one embodiment is a method for assigning nucleic acid sequence reads to target polynucleotides, which includes providing transposome complexes. In some embodiments, the transposome complexes include a transposase and a first polynucleotide having end sequences which can be used to fragment the target polynucleotides and insert into each fragment an end sequence or tag which can be used to bind to capture probes located on the substrate. The method can include contacting the transposome complexes with the target polynucleotides under conditions to fragment the target polynucleotides and add capture sequences to the ends of each fragment. In some embodiments, the capture sequences include P5 or P7 sequences as provided by Illumina, Inc. In some embodiments, the complexed strand and transposome is in solution, and is then brought towards a substrate and immobilized thereon. In some embodiments, prior to immobilization of the transposome complexes on the substrate, one or more of the transposome complexes bind the target polynucleotides in solution. In this embodiment, the transposome complexes in solution become immobilized to the substrate.

Once the fragments have been bound to substrate, the bound fragments can be amplified to form a plurality of nucleic acid clusters on the substrate. The location of each cluster on the flowcell can then be determined before, during or after performing sequencing by synthesis reactions (SBS) to obtain the nucleotide sequence of each fragment located in each cluster. Once the nucleotide sequence of each cluster has been determined, the method can start to map those reads to determine the original target polynucleotide from which the read originated. In some embodiments, the mapping process takes into account the flowcell location of each cluster, such that clusters which are closer to each other on the flowcell are more likely to have originated from the same target polynucleotide. In some embodiments, the library preparation steps are performed on the flowcell, which may reduce the complexity and the amount of equipment required for the systems. Furthermore, by mapping the sequenced fragments to target polynucleotides using the spatial information accompanying each cluster, the method performs more accurate mapping operations as compared to methods that do not take the spatial location of each cluster into account during the mapping process. Therefore, spatial information that includes relative distances between various clusters on a flowcell is leveraged to adjust mapping information, thereby increasing the read quality of previously identified multi-mapped reads. In the past, identified multi-mapped reads may have been discarded. Increasing the read quality of these previously discarded reads, by improving the confidence of read pair's alignment based on linking information with a high link quality score, may improve the alignment information and quality of information used in certain genomic analysis applications including, but not limited to, variant calling.

A relevant aspect to consider is the relationship between the area in the flowcell and the likelihood of having two fragments, which span a structural variant, land close to each other by chance. A small area in the flowcell reduces the probability of two fragments landing in close proximity due to the limited surface area to accommodate reads. Conversely, a larger area in the flowcell increases the likelihood of chance occurrences where fragments, including those from different chromosomes, land in close proximity. Consequently, utilizing a large threshold distance for read pairs in the flowcell to establish spatial links leads to an increased identification of spurious links. This new process of processing the DNA samples to create a collection of DNA fragments suitable for high-throughput sequencing (but where the distribution of the fragments is related to the original sequence of the longer original DNA) creates a difficulty in terms of defining the confidence that co-located fragments originate from the same molecule. Accordingly, it may be difficult to define (e.g. quantify and assess the quality) the relationship of fragments on a flowcell.

Embodiments described herein may address these issues by introducing a linking quality score, which has implications for various downstream applications like mapping, alignment, and variant calling. This link quality score not only enables the filtration of potentially erroneous reads but also aids in identifying high-quality links between fragments. As a result, the downstream processes become more efficient while also minimizing the computational memory required.

Another relevant aspect is the use of long range connectivity information to confirm or identify structural variants. The nature of structural variants themselves can make them difficult to detect with short reads since many structural variants affect large regions of the genome. Structural variants include deletions, insertions, duplications, inversions, and translocations that can range in size from a few base pairs to several megabases. When the size of the variant is near to or exceeds the length of the short reads, it becomes problematic to span the entire variant, and thus, deducing its presence and exact nature is more complex. Secondly, short read sequencing often employs processes that align sequence reads to a reference genome. If a structural variant is present, the short reads from that region may not align properly or at all to the reference. Additionally, repetitive regions in the genome exacerbate the challenges posed by short read sequencing. A significant portion of the human genome is composed of repetitive sequences. If a structural variant occurs within or near these repetitive regions, short reads may not provide unique alignment information. Determining the exact placement and context of such reads is challenging, leading to ambiguities in SV detection.

The introduction of long-range connectivity information in short read sequencing serves as an intermediary solution that bridges the gap between traditional short read sequencing and long-read sequencing in the context of structural variant detection. Firstly, the methods of the disclosure allow for the grouping of short reads that originate from the same, longer DNA molecule. This means that even if individual reads might be too short to span an entire structural variant, the collective information from a group of short reads can provide context about larger regions of the genome. When short reads are associated within a longer original DNA fragment, sequencing methods gain insight into regions of the genome much larger than the individual read lengths, thereby aiding in SV detection.

Secondly, the long-range connectivity information aids in resolving repetitive regions of the genome. By associating such short reads with others from a known anchor read or fragment, one can more confidently place these reads in their correct genomic context, reducing ambiguity and increasing the accuracy of SV detection. Additionally, having this extended context helps in the accurate reconstruction of the genomic landscape. This is particularly beneficial when dealing with complex structural variants or regions with multiple variants close together. Traditional short read methods might struggle to differentiate between such scenarios, but the added context from long-range connectivity can help disambiguate such scenarios.

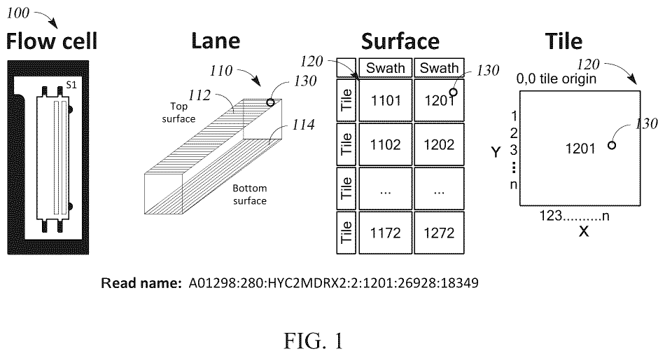

Referring now to FIG. 1, in some embodiments, a flowcell 100 that provides spatial information of read pairs includes a plurality of lanes 110. Each lane 110 includes a plurality of surfaces. As shown, in some embodiments of the flowcell 100, a lane includes a top surface 112 and a bottom surface 114. By way of example, if the reads being compared are on opposite surfaces, the distance between them is considered infinite because the assumption is that they cannot be linked. Note, however, that in some embodiments, it is possible that reads from different surfaces could be linked, especially as the size of the input template DNA molecule increases. In some embodiments, each surface is subdivided into a plurality of tiles 120. As shown, a cluster 130 may be located on a tile 120 that is designated as 1201. This designation serves as an illustrative example only and is not limited to the alphanumeric characters shown in the figure. In some embodiments, the tile 120 includes two-dimensional X-Y coordinates as shown to provide the spatial information between clusters. In some embodiments, the X-Y coordinates may be derived from information stored in a FASTQ file. In some embodiments, X-Y coordinates may be stored in or derived from a BCL (Base Call) file, which is a binary file format commonly associated with next-generation sequencing (NGS) platforms. In some embodiments, the x-y coordinates may be stored in an ORA file. DRAGEN ORA (Original Read Archive) compression technology is a lossless genomic compression technology that achieves very high compression ratios of FASTQ and FASTQ.GZ files especially on the latest Illumina sequencing platforms NovaSeq 6000, NextSeq 1000, and NextSeq 2000 systems: up to 5× ratio vs. gzipped FASTQ (FASTQ.GZ)

In some embodiments, the subdivision of the surface into tiles 120 is an artificial separation so that the surface of the flowcell is not separated into physical tiles, but instead the images captured by a camera can be segmented into tiles. As shown, the tiles 120 are subdivided into swaths, which roughly correspond to a pixel width of a camera used to capture images of the flowcell. In some embodiments, the tile 120 denotes the size of an image that can be captured by the camera. In some embodiments, the X-Y coordinates are pixel values. In some embodiments, 1 unit of a tile 120 can be approximated to be 1/10^{th} of a pixel. A physical separation is contemplated in some embodiments where the tile can have physical barriers, wells, and other structures which separate one portion of the flowcell from another portion of the flowcell. In some embodiments, spatial information, including X-Y coordinates, for clusters such as cluster 130 are obtained by a camera that processes the pixel value of the digital image.

One experiment that may provide spatial information on sequenced reads may be performed on a substrate having transposome complexes immobilized thereon. A transposome complex may include a transposase and a first polynucleotide including an end sequence and a first tag in some embodiments. The sequencing experiment may proceed by contacting the transposome complexes with target polynucleotides under conditions to fragment the target polynucleotides. The fragmented target polynucleotides may then be amplified to form a plurality of nucleic acid clusters on the substrate. The plurality of nucleic acid clusters on the substrate are microscopically observable and their location data may be recorded. After the location information has been obtained, then the nucleic acid sequence reads of the fragmented nucleic acids may be sequenced and the corresponding location data may be stored.

In some embodiments, a functional definition of “near” indicates that the sequence reads originate from the original template. Variably this may mean that near mean within a threshold distance of 10,000 nm, 5,000 nm., 3,000 nm., 2,000 nm, and 1,000 nm. In some embodiments, nearby may mean within a certain number of proximate wells. For example, on a substrate which includes wells for each read cluster, the number of wells between clusters may be much greater than 50, than 100, or than 200 wells. In some embodiments, nearby may depend on x/y direction as the diffusion pattern may not be uniform after fragmentation. For example, the links may form an oval pattern on the flowcell.

Described herein are systems and methods of establishing link quality scores for the links determined between read pairs based on spatial information obtained on the flowcell. This spatial information may be, for example, the cartesian coordinates of the cluster which contains a particular read on the flowcell. The spatial information may include a location of a well on a substrate in one embodiment. To establish these spatial links between two reads, in one embodiment two thresholds are used. The first is the spatial distance threshold, which represents the physical distance between two reads on the flowcell.

In some embodiments, the spatial distance may be measured in nanometers. In some embodiments, the spatial distance may be measured in a unit of length relative to the flowcell. For example, a flowcell unit may be relative to the size and/or spacing of patterned clusters on a flowcell. In some embodiments, two differently patterned flowcells may have different absolute units of length due to different density of clusters on the surface. In some embodiments, the spatial distance may be an absolute unit of length, or any other unit of length consistent with the disclosure. In some embodiments, the spatial distance may be included in a FASTQ file, which generally is a text file that contains the sequence data from the clusters that pass filter on a flowcell. FASTQ files can be used as sequence input for alignment and other secondary analysis software.

The second threshold is a genomic distance threshold, representing the distance between the two reads on the genome after mapping. In some embodiments, a genomic distance may be based on a reference genome. In some embodiments, other methods may use distance in a sample genome. An empirical method for establishing thresholds will vary widely between experimental conditions. This disclosure provides for methods to attach a link quality score to a link as a factor of the spatial and genomic distance between two potentially linked reads. As described in more detail below, one method of determining the quality of a link between two reads is to estimate the null distribution of pairwise read pairs. This null distribution can provide the basis for calculating the “false discovery rate”, which can then be used as a proxy for the link quality score of the link.

A linking quality score is defined as a numerical representation that quantifies the reliability of a link between two read pairs. This score may be calculated using multiple metrics that contribute to the quality of the link, and the linking quality score may serve as a composite measure that simplifies complex relationships into a single, easily interpretable value.

A linking quality score may provide a basis for comparison or decision-making. For example, a high linking quality score between two read pairs might indicate that two reads are highly likely to originate from the same DNA fragment, and thus should be paired for further analysis, but also that the conditions used to generate that link may be tuned and evaluated on the basis of the score. The formula for calculating a linking quality score may vary, and could be determined based on a false discovery rate, a metric quantifying type II error, a weighted average of different contributing metrics, and a machine learning model trained to predict link quality based on multiple features. In either case, the linking quality score aims to encapsulate diverse considerations into a single number representing a link's overall “quality,” thereby facilitating quantitative analysis.

In preparation for reading sequences from a flowcell, a DNA sample may be obtained, and the DNA is then fragmented so that short fragments are used to generate clusters on the flowcell. Flowcells are specialized glass slides with a chemically treated surface designed to capture and immobilize DNA fragments via adaptors. The loading process itself might be tuned so that spatial localization on the flowcell mirrors the original proximity of fragments in the polynucleotide. This could be achieved by carefully controlling the flow rates, concentrations, and temperature during the loading process. Specialized techniques such as ‘gradient loading,’ where DNA concentration varies across the flowcell, might be used to enhance this effect.

Fragmenting polynucleotides such as DNA or RNA by using transposases bound to a flowcell differs from traditional enzymatic or mechanical methods. In a typical setup, a flowcell with immobilized transposases is prepared. As the polynucleotide sample flows through the cell, the transposases cut the DNA or RNA at specific or random sequences and may optionally insert short adapter sequences. Transposomes are complexes formed by a transposase enzyme and a short piece of DNA known as a transposon. In the context of fragmenting polynucleotides like DNA, the transposome performs two main actions: it cleaves the DNA at specific or random locations and may simultaneously insert a transposon sequence. This process is often referred to as “tagmentation.” This process occurs in situ, or directly on the flowcell, negating the need to remove the sample for separate fragmentation steps.

Transposomes function to cut DNA and insert adapter sequences, but typically do not serve to anchor these fragments to a surface. Flowcells are generally prepared to bind DNA or RNA fragments through specialized adapter sequences, often after the library preparation process has already been completed. In this common arrangement, the DNA would be first fragmented and go through library preparation, including the ligation of appropriate adapter sequences, before being loaded onto a flowcell for sequencing.

In some embodiment, however, transposases may be immobilized on the surface of a flowcell, designed to perform fragmentation in situ as the DNA flows through. Chemical functionalization may be added to the transposon sequences, allowing them to bind to the surface of the flowcell immediately upon insertion. This would mean that the transposase would not only cut and tag the DNA with an adapter but may also anchor it in place for subsequent sequencing. In some embodiments, alternate methods may be used to bind the fragments with adapter sequences that are separate from the transposome.

In a case where the polynucleotide is fragmented after loading onto the flowcell, library preparation could be modified accordingly. Traditional library preparation involves several steps before loading onto the flowcell, such as fragmentation, end-repair, adapter ligation, and sometimes amplification. When the polynucleotide is fragmented in situ, certain methods according to the disclosure may skip the fragmentation and possibly even the adapter ligation steps before loading, depending on the design. After fragmentation on the flowcell, the library could be immediately prepared for sequencing. If adapters are not already added by the transposases, they may be introduced by flowing adapter molecules through the cell under conditions that favor ligation.

Once the DNA fragments are immobilized on the flowcell, the sequencing process begins. Most modern sequencing platforms use a method known as “bridge amplification” to create clusters of identical DNA fragments on the flowcell. This is followed by the actual sequencing step, where nucleotides are added and their incorporation is detected, thereby generating the sequence reads. The end result is sequence reads from the flowcell where fragments of the polynucleotide that were proximal in the original structure also have a higher likelihood of being sequenced as adjacent or nearly adjacent reads. This spatial correlation can significantly aid downstream data analysis, especially in applications like detecting structural variations, assembling genomes, or reconstructing haplotypes.

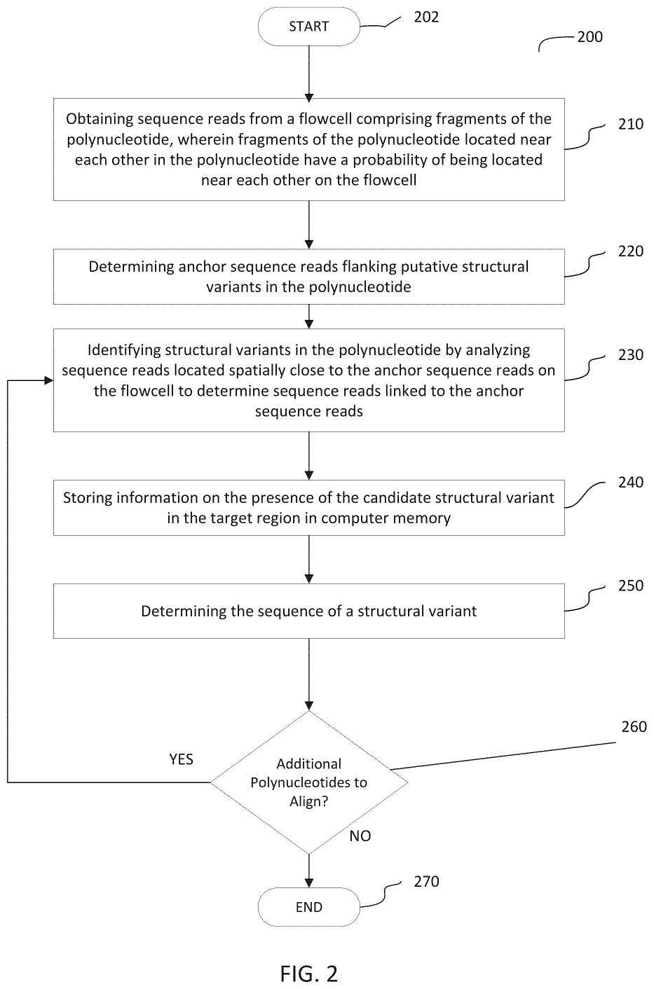

FIG. 2 is a flowchart of an example method 200 for identifying structural variants in a polynucleotide. The process of obtaining sequence reads from a flowcell that contains fragments of a polynucleotide—while ensuring that fragments located near each other in the original polynucleotide have a higher probability of being proximate on the flowcell—may entail the following steps.

The process 200 begins at a start step 202 and then moves to a step 210 wherein the method includes obtaining sequence reads of fragments of a polynucleotide wherein fragments of the polynucleotide located near each other in the polynucleotide have a probability of being located near each other on the flowcell. In some embodiments, the method may include various methods of obtaining sequence reads during or after performing a sequencing experiment. In some embodiments, the obtained reads may be filtered to only include read fragments located near each other on a flowcell. In some embodiments, fragments of the polynucleotide located near each other in the polynucleotide may be retrieved before or after an alignment step. Consistent with the disclosure, methods of fragmenting polynucleotides on a flowcell cell may produce fragments of a polynucleotide where the probability of the fragments being located near each other on the flowcell may be correlated with a genomic distance between the fragments in the original polynucleotide molecule.

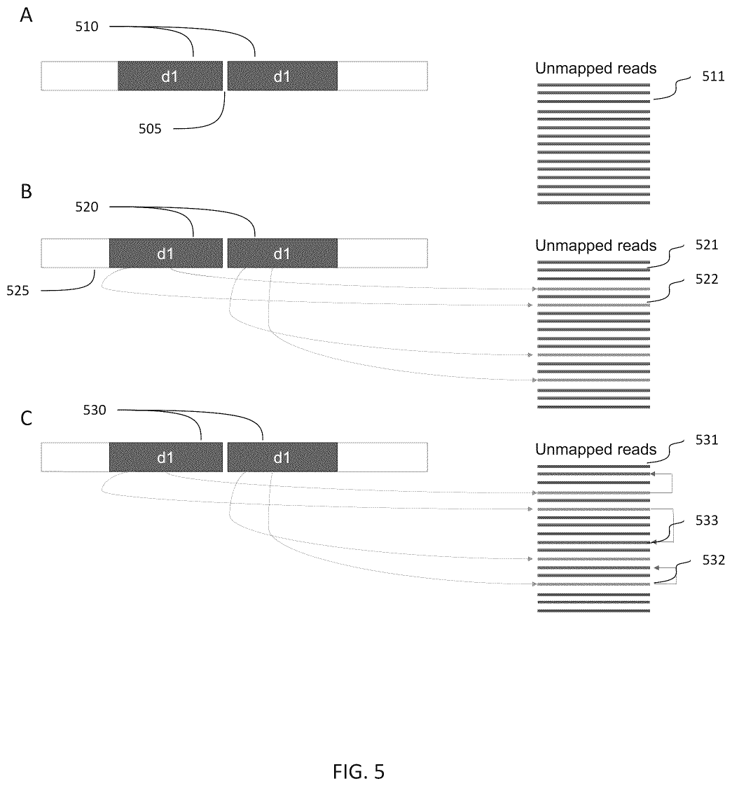

As described above, sequence reads derived from a fragment on a flowcell are then assigned to a specific location on a reference genome where the sequence read was detected. Some of reads will align with a high MAPQ to a sample genome or a reference genome, meaning that there is a relatively high likelihood that the sequence read actually was derived from that position on the reference genome. These reads, having a relatively high MAPQ may be used within embodiments as anchor reads. Thus, once the method 200 obtains sequence reads at the step 210, the process 200 moves to a step 220 wherein the process determines anchor sequence reads flanking putative structural variants in the polynucleotide. In some methods of structural variant detections, one read in a read pair might serve as the anchor read while the other spans a structural variant. For example, at step 220, the method may proceed by determining anchor sequence reads, such as in a region 525 in FIG. 5, which may be known to flank putative structural variants (e.g., structural variant breakend 505) in the original polynucleotide molecule or reference genome. In some embodiments, the genomic distance between a spatially linked read and an anchor read may vary. In some embodiments, an anchor read may flank a structural variant at any distance where the linked read spans at least a portion of the structural variant.

Anchor reads are usually characterized by a high degree of similarity to known sequences in the reference genome, often facilitated by processes that assign high-quality alignment scores based on the number of matches, mismatches, gaps, and other criteria. In general, these are reads that can be mapped unambiguously with a high MAPQ to unique positions in the reference genome, making them useful for subsequent analyses, such as the identification of structural variants. In some embodiments, a spatially linked anchor read may later serve as an additional anchor read for other sequence reads if the spatially linked anchor read is mapped to the reference genome.

The quality and reliability of anchor reads is important, as they set the basis for further analyses. The confidence in these reads is typically quantified using measures like MAPQ (Mapping Quality) and but may also use other metrics such as an alignment score, coverage, or uniqueness of alignment to determine the confidence in using a read as an anchor read. For example, a MAPQ score of 60 might indicate very high confidence, whereas a score of 0 indicates no confidence. In some embodiments, a threshold MAPQ score for an anchor read may be any of 50, 40, 35, 30, 25, 20, 19, 18,17, and 15. In addition or as an alternative, reads that align uniquely to one location in the genome are generally more reliable than those that can align to multiple locations (multi-mappers). The ability of a read to align uniquely can be a criterion for considering it as an anchor. In other cases, the presence of known single nucleotide polymorphisms (SNPs) in a read can be used to assess its reliability. Reads that contain SNPs that are consistent with a reference database might be seen as more reliable.

As each of the sequence reads has information about the spatial location of the reads, groups of sequence reads that are near each other may be identified. For example, after identifying anchor reads at step 220, the process 200 moves to a step 230 wherein method 200 identifies structural variants in the polynucleotide by analyzing sequence reads located spatially close to the anchor sequence reads on the flowcell to determine sequence reads linked to the anchor sequence reads. This process may analyze sequence reads located spatially close to the anchor sequence reads on the flowcell, and determine if any of these proximate reads are unmapped. The method may identify structural variants in the polynucleotide by determining sequence reads linked to the anchor sequence reads. Accordingly, some embodiments may rescue unmapped reads, whereby reads that were not able to be used in an assembly or alignment process, because the reads, for example, mapped ambiguously to multiple regions, might be mapped to a unique position and used in an assembly/alignment process. Some embodiments may reduce the false positive rate by reevaluating whether an alignment for a read is correct by determining if there are the corresponding links between two proximate sequences. In some embodiments, a structural variant may be identified in a region of a reference genome without any initially mapped reads, but where spatially linked reads may indicate that a region should map to a particular part of the genome. In some embodiments, the region with the putative structural variant may already have mapped reads. Consistent with the disclosure, anchor reads may be used to identify candidate structural variants. In some embodiments, the presence of a structural variant may be stored with or without determining the sequence of the structural variant. In some embodiments, the method may store the sequence of the structural variant based on the sequence of the spatially linked sequence read. In some embodiments, a proximate read may have an incorrect mapping, such as to a highly repetitive region with a single point mutation, and the methods may determine that the read is linked to an anchor read at a different location in the genome indicating that the proximate read is either potentially misaligned or potentially in a region spanning a structural variant.

After the presence of a candidate structural variant is detected at step 230, the process 200 moves to a step 240, where the method stores the detected information regarding the presence or absence of the candidate structural variant within the target region in computer memory. Determining the presence of a structural variant in a genomic sequence begins with detection, and accordingly the method may store detected information such as a flag that there is a putative structural variant at a location in the reference genome. In some embodiments, a scoring value associated with the mapping of a sequence read may be updated to indicate the presence of a structural variant. In some embodiments, the step of detecting structural variants may be combined with various methods of determining the sequence of the structural variant. Storage facilitates future analyses and serves as a record for verifying and validating the detected structural variants. For example, after storing the candidate structural variant at step 240, the method 200 moves to a step 250, where the stored information may be optionally used to confirm the nucleotide sequence of the candidate structure variant.

After the structural variant is detected a decision may be made at a decision step 260 whether there are additional polynucleotides to align. If additional read pairs are left unmapped, or there is any other indication that there would be additional undetected structural variants, the process 200 may loop back to step 230, where additional structural variant may be detected. In some embodiments, the process may repeat at step 220, where additional anchor reads are determined, by for example establishing a contig, before proceeding to step 230 again. If there is no further need to detect additional structural variants, the method may conclude at step 270.

Consistent with the disclosure, the genomic data referenced in the previous steps may be obtained by various methods, whether indirectly from databases, or pre-processed information, or from a sequencing system and any associated raw data. For example, one way to acquire genomic information referenced in step 210, may be by retrieving it from local or remote databases. These databases may store genetic data from various sources, including genomes, genes, sequences, and annotations. In some cases, genomic information may be pre-processed and shared directly. This pre-processed data could include aligned reads, variant calls, or other specific genomic analyses.

Genomic information may also be obtained directly from a sequencing system. The sequencing system may generate raw data in the form of DNA sequence reads, and the corresponding pixel or location where that sequence read was sequenced. These reads can then be processed using alignment process to map them to a reference genome, identify variations, and reconstruct genomic sequences. This may involve intermediary steps like quality control, removing adapter sequences, and trimming low-quality bases. In some embodiments, alignment process may be applied before or after such steps and may be iteratively applied to map the reads to a reference genome. In some embodiments, the system may map the reads, allowing for downstream analyses such as variant calling or structural variant identification.

The data obtained from spatially linked read pairs may be distinct from that of, for example, barcoded read pairs due to the way information is captured and utilized. Spatially linked read pairs may involve associating the physical positions of DNA sequences on a sequencing substrate. This means that the data provides insights into the two-dimensional placement of genetic material on a sequencing substrate. This information can be valuable for understanding whether different read pairs came from a single molecule. On the other hand, barcoding read pairs typically involves adding short DNA sequences (barcodes) to the DNA fragments before sequencing. These barcodes serve as molecular “tags” that help distinguish and track different DNA fragments from the same source. The primary purpose of barcoding is often to associate related reads, ensuring they come from the same genomic template. Source information and proximity information for read pairs relate to the relationship between two reads, but they focus on different aspects.

Source information refers to the origin or source of the two reads within a read pair. In other words, it indicates which DNA template or genomic region the two reads were derived from. This information may be used to correctly associate reads that are part of the same genomic fragment or template. Source information is typically obtained through barcoding or other labeling methods. For example, each DNA fragment might be assigned a flag before sequencing, so when two reads share the same flag, it means they come from the same original DNA molecule or template.

Proximity information, on the other hand, relates to the physical closeness or distance between the two reads within a read pair. This information is particularly relevant when reads are generated from spatially arranged templates, such as in spatial transcriptomics or spatial genomics. Proximity information indicates that the two reads were captured from nearby physical locations on a substrate or within a tissue. This information provides insights into the spatial relationships and organization of genetic material, revealing how different genomic elements are positioned relative to each other. While both source and proximity information may be associated with read pairs, they may serve different purposes. Source information helps correctly link reads that belong to the same template, while proximity information provides insights into the local connectivity of read pairs. In some embodiments, these two types of information might be used together to better identify structural variants.

### Detection of Structural Variants through Read Alignments

The processor may be equipped with capabilities for identifying putative structural variants by analyzing variations in the alignment of sequence reads compared to a reference genome. By doing so, the processor may effectively identify discrepancies that could be indicative of structural changes in the genome. As an alternative, the method may be designed to highlight potential structural irregularities by examining differences in how sequence reads map against a consensus genome.

The introduction of the disclosed method for variant detection represents a significant advancement in the field of genomics, offering enhanced capabilities for identifying structural variants that were difficult to detect with traditional techniques alone. This new method leverages the method's capability to scrutinize the alignment of sequence reads spatially around anchor reads with a reference genome or sample genome. By doing so, the disclosed methods have the potential to flag a wider range of genomic structural changes, filling in the gaps left by existing methods.

When used in conjunction with established approaches, the new method serves as a complementary tool that may augment the overall efficacy of a variant detection process. Traditional methods, often reliant on single-nucleotide polymorphism (SNP) analysis or simpler alignment techniques, can excel in identifying certain types of genetic variants but generally fall short when complex structural rearrangements are involved. The disclosed systems and methods may corroborate findings from these traditional methods while also uncovering variants that might otherwise go unnoticed.

For example, the processor in the new method may also retrieve reads that may be spatially close to anchor reads, thereby offering a more localized context that could be useful for confirming variants identified by other methods. Some embodiments may retrieve unmapped reads that are within a threshold distance to the anchor reads. The processor's ability to assemble these nearby unmapped reads into a contig sequence for further analysis is yet another advantage. This particular feature allows for more granular examinations of the genome, potentially revealing structural changes that simpler methods could miss. In some embodiments, a processor is configured to assemble the retrieved unmapped reads into a contig sequence.

The method may iteratively proceed by searching for read sequences spatially proximate to anchor sequence reads. In some embodiments, the anchor sequence may be a read mapped with high confidence. The anchor sequence read may be a read mapped with a MAPQ score of at least 20. The anchor sequence may be applied in context of paired end sequencing. For example, the anchor sequence read may be a paired end read that aligns to a reference genome. In some embodiments the system may proceed by detecting a putative structural variant from variations in read alignments between a reference genome and the sequence reads.

The method may assemble the contig sequence by, for example, constructing a de Bruijn graph from k-mers of the retrieved unmapped reads. This method could offer a more robust and accurate assembly of the sequence. Note that in addition or in parallel with the methods of the disclosure, the method may also assemble the contig sequence by constructing a de Bruijn graph from k-mers of the retrieved reads. The process of assembling a contig sequence using a de Bruijn graph begins with the extraction of k-mers from the set of retrieved unmapped reads. A k-mer is a contiguous subsequence of length ‘k’ taken from the read. For example, if the read is “ATCGAT” and k is 3, then the possible 3-mers would be “ATC,” “TCG,” and “CGA.” In some embodiments, the methods may be used in combination with K-mer frequency analysis, which is a method in nucleotide sequence analysis that can be used to Estimate biases, repeat content, and sequencing coverage.

Once the k-mers are extracted from all the unmapped reads, the next step is to construct the de Bruijn graph. In this graph, each unique k-mer serves as a node. Edges are drawn between nodes (k-mers) that overlap by (k−1) bases. To illustrate, if k is 3, and we have two 3-mers: “ATC” and “TCG,” they would be connected by an edge since they overlap by “TC,” which is (k−1)=2 bases long. The de Bruijn graph may be used to generate the structure of the sequence, where multiple k-mers will overlap in areas where the sequence is conserved. The complexity of the graph will vary depending on the diversity of k-mers, which is in turn influenced by the original sequence complexity, including any repeating elements or structural variations.

Once the de Bruijn graph is constructed, the next step may be to identify paths within the graph that represent legitimate sequences. This may be done using graph processes that seek to find Eulerian paths, which traverse each edge exactly once. The sequence of k-mers along an Eulerian path constitutes a contig, a contiguous sequence that approximates a region of the original genome. In cases where Eulerian paths are not feasible due to the graph's structure, alternative methods such as Hamiltonian paths may be considered. After constructing the contigs, additional steps may include error correction, gap-filling, and possibly scaffolding with other types of data to build longer, more accurate sequences. The assembled contigs provide valuable information about the genomic regions represented by the initially unmapped reads.

In some embodiments, a processor may be configured to align anchor sequence reads, and retrieve reads, before performing assembly. In some embodiments, a processor may be configured to align anchor sequence reads, and retrieve reads, after performing assembly. In some embodiments, a processor may be configured to identify structural variants in the polynucleotide by analyzing sequence reads located within a threshold distance to the anchor sequence reads on the flowcell to determine sequence reads linked to the anchor sequence reads.

Furthermore, the use of advanced techniques such as de Bruijn graph-based assembly for creating contig sequences enhances the accuracy and reliability of the variant detection. This multi-layered, more comprehensive approach to variant detection enables researchers and clinicians to generate a richer, more complete picture of an individual's genomic landscape. The new method for variant detection may also be designed to operate without relying on a reference genome, a departure from traditional techniques that depend heavily on such reference points for alignment and variant calling. By doing so, this method broadens its applicability and may improve its ability to detect novel structural variants that do not align well with known reference genomes.

In the absence of a reference genome, the processor may employ de novo assembly techniques to generate contigs, or contiguous sequences, from the raw sequence reads. These assembled sequences serve as a stand-in for a reference genome, offering a framework upon which to identify variants. The use of de novo assembly allows the method to be more adaptable and could be especially useful in studying organisms or genomic regions that have not been well-characterized. Additionally, this method may also utilize graph-based approaches to represent the multiple possible configurations of a genomic region. Graph-based structures such as de Bruijn graphs can help capture the complexity of the genomic landscape without forcing it into a linear, reference-based mold. This is particularly important for detecting structural variants that may involve complex rearrangements or repetitions that a reference genome might not adequately represent.

The ability to work without a reference genome opens the door to more flexible analyses. For example, the processor could still retrieve unmapped reads that may be spatially close to what the process identifies as anchor points within the de novo assembly. These reads could then be incorporated into further assemblies, thereby enriching the genomic representation and enhancing the detection of structural variants. Furthermore, operating without a reference genome may allow for a more unbiased detection of variants, reducing the risk of missing variants that are not present in the reference.

Once the method detects a candidate structural variant, the candidate may be evaluated based on various target and baseline metrics. A significant divergence between these two metrics would indicate the potential presence of a structural variant in the target region, warranting further investigation. A baseline metric may be generated, for example, by examining spatially linked read pairs in a genomic background region that is not expected to harbor structural variants. The baseline metric is useful because the complementary information of spatial links may vary from sample to sample, and does not necessarily include any universal characteristics that may be applied versus a target sample. For example, the number of spatial links may naturally decrease for sequences near the end of a chromosome. Accordingly, a baseline region may be selected for a comparable region near the end of a chromosome, which provides an appropriate number of inbound and outbound links for read pairs near the end of a chromosome.

A target metric can be compared with the baseline metric through a statistical comparison. The comparison could employ simple statistical tests, such as a t-test or chi-square test, or more complex statistical models, such as logistic regression or machine learning processes, depending on the complexity of the data and the specificity and sensitivity required. Regardless of the statistical method employed, the focus remains on determining whether the target metric diverges from the baseline metric. By way of example, a divergence may be quantified by a threshold count of links, a threshold average number of links, or a standard deviation in the length of links. A divergence between the target and baseline metrics could serve as a robust indicator of a structural variant in the target region. This is because the baseline region, having consistent numbers/lengths of links between reads and without expected structural anomalies, provides a ‘normal’ genomic environment without structural variants against which the target region can be contrasted. Consequently, anomalies in the target region, reflected as deviations from the baseline, become highlighted, indicating the likely presence of structural variants.

An aspect of the disclosure is directed to physical flowcell information from “pileups” of physically close fragments that are shown to align at far genomic locations as evidence of SV. For example, sequence reads with similar X-Y coordinates on the flowcell, while their genomic alignment distance is “far” away is a strong indication of the presence of a structural variant. Quantifying “far” in terms of genomic distance could vary based on the organism's genome size and complexity. Generally, ‘far’ might mean that the reads align to different chromosomes, or to locations within a chromosome that are several kilobases (kb) or even megabases (Mb) apart. The exact definition “far” would depend on the specific thresholds. Structural variants in a genome can span a wide range of distances, from just a few kilobases (kb), such as 5 kb to 10 kb, to much larger scales, reaching several megabases (Mb), like 1 Mb to 5 Mb, depending on the specific nature of the genomic alteration.

Accordingly, the disclosure provides for methods of identifying genomic variants in a polynucleotide with the following steps. As described above, methods may start by obtaining genomic data comprising polynucleotide sequence reads and coordinates of the polynucleotide sequences from the polynucleotide on a sequencing substrate. The method may proceed by then aligning the polynucleotide sequence reads to a reference genome. Then the method may select aligned polynucleotide sequence reads which are within a predetermined distance from one another on the sequencing substrate. This subset of reads may be used to determine a genomic distance between the alignments on the reference genome of the aligned polynucleotide sequence reads with the selected polynucleotide sequence reads. Then the method may identify a polynucleotide as having a candidate genomic variant, when the aligned polynucleotide sequence reads are near each other on the flow cell within the predetermined distance and have a genomic distance above a calculated value.

### Determining Structural Variants

In addition to the process described above, the step of comparing a baseline region of a genome to a region containing a structural variant may use various metrics to quantify differences between these two regions. For a population-level comparison, metrics such as the total number of supporting links within a given region may be used. This metric would represent the count of connections observed between short reads or long-range signals in a specific genomic area. In a baseline region, one can expect a certain range of link counts that reflect the typical genomic connectivity. In contrast, a region containing an SV might exhibit a significant deviation from the link count, in the baseline region signaling the presence of a structural variant.

Another metric may be the average length of the links within a region. This metric characterizes typical lengths between the genomic connections for a given region. Deviations from the baseline average length of links in a region with an SV can indicate changes in the physical arrangement of the genome, such as insertions or deletions.

The distribution of link lengths within a genomic region may also offer insights into the presence of a structural variant. Metrics like the skewness and standard deviation of this distribution may be used to quantify the extent of departure from the expected link lengths in a baseline region. These metrics might exhibit pronounced distribution shifts in a region containing an SV, indicating altered genomic architecture, i.e., a structural variant.

The cumulative distribution function (CDF) of link lengths is another useful metric. It provides a comprehensive view of how the link lengths are distributed across the region. Deviations from the baseline CDF in an SV-containing region can highlight variations in the genomic structure that might correspond to specific types of SVs, such as insertions or deletions.

To account for population variability, statistical significance tests can be employed to compare the metrics of baseline and SV-containing regions. Hypothesis tests, such as t-tests, can ascertain whether the observed metric differences are statistically significant, providing a means to evaluate the detection of SVs.

In summary, a range of metrics may be employed to compare baseline genomic regions with regions harboring structural variants. These metrics encompass population-level measurements, average and distribution analyses, and statistical tests, collectively offering a comprehensive perspective on the alterations induced by SVs. By interpreting these metrics, researchers can unravel the intricate genomic changes brought about by structural variants and infer their potential functional implications.

### Retrieval of Spatially Close Reads

In some embodiments, the method may also retrieve reads that may be spatially close to the anchor reads. For example, the method may be configured to fetch unmapped reads that may be proximal to the anchoring reads. By doing so, the methods of the disclosure may add an extra layer of information that is specific to the region being studied, potentially increasing the accuracy of structural variant detection.

Unmapped reads in genome sequencing can arise for various reasons, and they can be broadly categorized into different types based on the characteristics of their sequence and alignment. One prominent type of unmapped reads is the Ambiguously Mapped Reads. These specific reads may be characterized by their potential to align equally well to multiple locations within the reference genome. The intrinsic sequence quality of these reads is often high, indicating that the sequencing process was successful and reliable. However, the challenge arises during the alignment phase. The alignment process, despite the high quality of the read, may find it challenging to assign these reads a definitive position within the genome. This ambiguous nature of alignment is typically observed in genomic regions known for their repetitive sequences. It's essential to approach these reads with caution, recognizing their inherent value and not hastily discarding them. They can still provide pivotal information, especially when studying genomes with high repeat content or when analyzing evolutionary patterns where repetitive elements play a role.

Target reads can sometimes be selected from the pool of Unmapped reads, which may be reads that do not align to any specific location in the reference genome. The idea behind targeting these Unmapped reads is that they may contain novel or rare sequences that may be not represented in the reference genome. These sequences could be of particular interest in discovering new genomic elements or identifying specific mutations that have not yet been characterized.

Another category of reads that might be targeted for further analysis are those that could be Randomly Mapped Incorrectly. These are reads that have been aligned to the genome but may be suspected to be in the wrong location. This misalignment often occurs in regions with common repeats, where the alignment process has difficulty accurately placing the read due to the presence of multiple, similar sequences in the genome. In some cases, these incorrectly mapped reads can be retrieved for reanalysis by examining their unmapped paired-end mates, which can provide clues to their correct placement. By targeting these reads, researchers can often gain insights into the architecture and function of repetitive elements in the genome.

In some embodiments, target reads may be sourced from reads that may be mostly mapped incorrectly, often as a result of a duplication event in the genome. These reads partially align to the reference genome but are primarily positioned in an incorrect location due to the confusing presence of a duplicated sequence elsewhere. As with Randomly Mapped Incorrectly reads, these can sometimes be corrected by analyzing the alignment patterns of their paired-end mates.

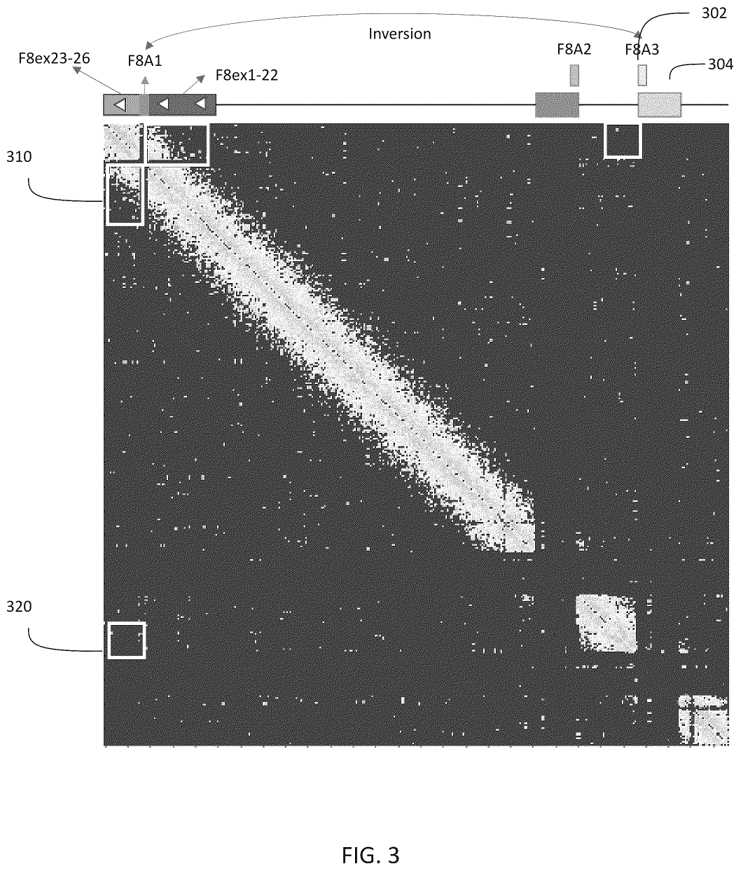

FIG. 3 displays a colocation heatmap that shows the relationships between linked read pairs in the Factor VIII gene. On the heatmap, the X-axis and Y-axis correspond to the genomic coordinates of the gene. The starting point of the gene is situated at the top-left corner, progressing to the gene's other end at the bottom-right. Above the colocation heatmap is a cartoon representation of the gene under study, which serves as a guide for interpreting the heatmap below. This cartoon outlines the gene's structure and highlights the relevant regions, making it easier to locate these areas on the heatmap. Different colors in the cartoon symbolize various gene features. Orange blocks 302 indicate 10 kbp segmental duplications (segdups) that are identical to each other, with three copies represented. Green blocks 304 signify 50 kbp segdups that are also identical. Apart from these, the heatmap is labeled to highlight specific regions, including F8ex23-26, F8A1, F8ex1-22, and F8A3.

One notable feature from the heatmap is the presence of areas with a high density of links, which usually occur where read sequences are located near to adjacent sequence reads on a flow cell and aligns to the reference genome at a position adjacent to the sequence read. These high-density areas appear darker or more intense on the heatmap, indicating a higher frequency of linked read pairs. On the other hand, lighter or less intense areas are seen where read sequences are genetically distant from each other, such as between different segmental duplications or between non-adjacent exons. These regions have fewer linked read pairs, demonstrating that distant genetic sequences are less likely to be linked together.

The heatmap serves as an informative tool for understanding the relationships between different regions within the Factor VIII gene. For example, boxes highlight specific areas of interest in the figure. Box 310 clearly shows a large number of connections between the F8ex23-26 and F8ex1-22 regions, as evidenced by the dark or intense coloring within this box. This suggests that these two exonic regions are closely related in terms of genomic architecture, often appearing together in linked read pairs. Another box 320 draws attention to the complete lack of connections between the F8ex23-26 region and the area upstream of F8A3. The color within this box is notably lighter, signifying the expected absence of linked read pairs between these two remote regions.

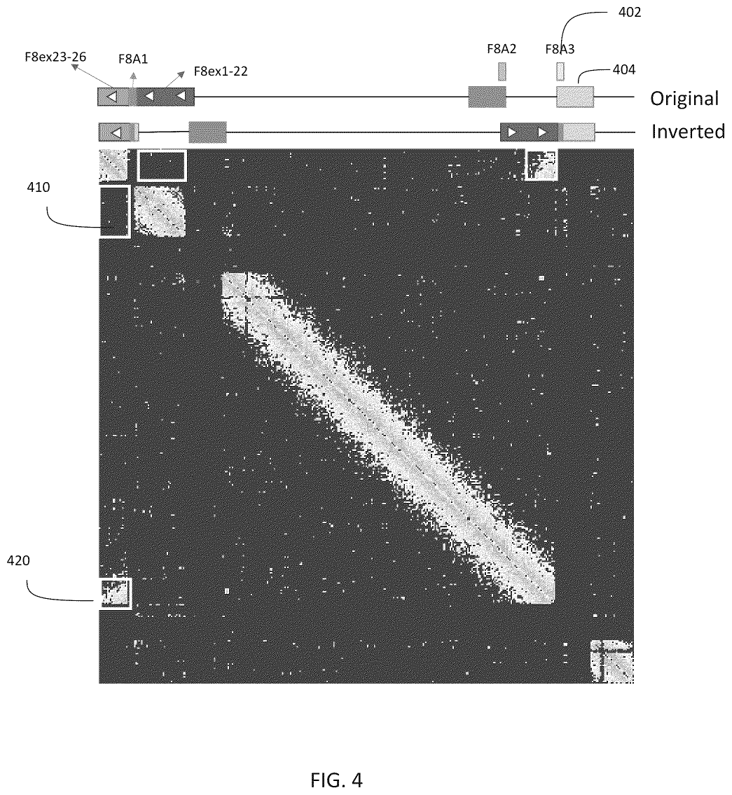

FIG. 4 displays a colocation heatmap representing the relationships among linked read pairs across different regions of a gene, believed to be a version of the Factor VIII gene. As in the previous figure, the genomic coordinates of the gene extend along both the X-axis and Y-axis, with one end of the gene located at the top-left corner and the other at the bottom-right. Different colored blocks, such as orange for 10 kbp segmental duplications and green for 50 kbp segmental duplications, are again used to indicate specific genomic features. Regions such as F8ex23-26, F8A1, F8ex1-22, and F8A3 are again labeled.

Box 410 highlights an area showing no connections between the F8ex23-26 and F8ex1-22 regions. This is illustrated by a redder or less intense color within this specific box. This lack of connectivity between these exonic regions is different from what is usually seen in a standard gene structure, suggesting an anomaly in this particular gene's architecture.

Box 420 focuses on a number of new connections between the F8ex23-26 region and the areas upstream of F8A3. This is shown by a darker or yellow/green color within the designated box. Such connectivity between these two regions is unusual and points to a rearrangement in the genomic structure.

The new connections between F8ex23-26 and the region upstream of F8A3, coupled with the absence of links between F8ex23-26 and F8ex1-22, strongly suggest the presence of an inversion structural variant within this version of the Factor VIII gene. Typically, inversions result in unexpected linkages between regions that are otherwise distant or unrelated in the canonical gene structure. The presence of these unusual links provides compelling evidence for the existence of a structural variant, specifically an inversion, in the gene represented in this heatmap.

Overall, this figure offers a contrast to the earlier heatmap and provides clues pointing to an alteration in the gene's architecture. The heatmap serves as an example of how links between collocated genes may be used as a powerful diagnostic tool for detecting structural variants, aiding in further research and possibly having implications for the understanding of diseases like hemophilia A that involve mutations or alterations in the Factor VIII gene.

FIG. 5 illustrates an example of a process of identifying subpairs linked to breakpoints in genomic data. The sequence of steps, from initial conditions to the recursive ‘rescue’ operations. The figure includes three distinct panels A, B, C, each illustrating a different step in the process. The process involves both mapped (anchored) and unmapped reads, demonstrating how they interact at different stages of the process to iteratively reveal additional subpair links. In the first panel, panel A, a region of reads, labeled as ‘d1’ 510, is introduced which is positioned to each side of a breakpoint 505. Alongside, a number of unmapped reads 511 is also shown, representing the initial state of the data before the process begins.

The panel B, located directly below the first, demonstrates an example process that searches for potential links between the anchoring reads from the region of reads ‘d1’ 520 and the group of unmapped reads 521. The panel graphically represents how the process identifies these links between reads at 520 and reads nearby in the genome 525, potentially forming connections between reads that were initially separate. This panel provides a snapshot of the process's ‘linking’ phase, serving to elaborate how the method goes about identifying further connections in the dataset. Once the link is formed, the unmapped read 521 may be aligned to a location in the genome and become rescued reads 522.

The panel C at the bottom of the figure shows the iterative nature of the process. It shows that the newly identified set of ‘rescued’ reads 532 may now be used to discover additional linked reads 533 within the group of unmapped reads. Essentially, this panel illustrates the recursive aspect of the process, emphasizing that the process may be repeated either until no more reads can be rescued or until sufficient coverage for the relevant genomic region has been achieved. In some embodiments, because the structural variant may cause alignment issues, the unmapped reads 531 may have reason to be linked with a number of other unmapped reads 531. Accordingly, once a first unmapped read 531 is aligned as a rescued read 532, the rescued read may link to additional linked reads 533.

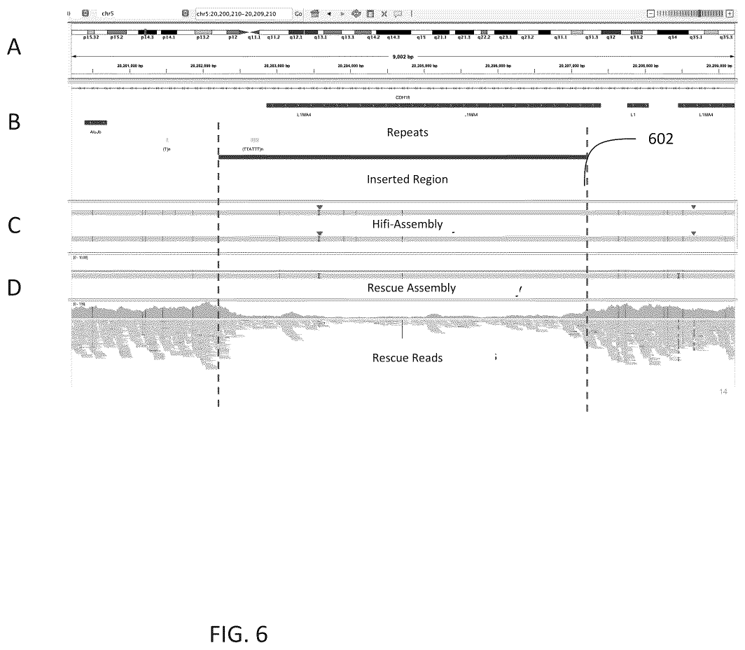

FIG. 6 presents a multi-layered alignment plot which illustrates the alignment of reads to the Human GRCh38/hg38 reference genome within a specific genomic region. The plot is organized into several stacked panels, and shows how the disclosed methods may improve the alignment, assembly and number of false negative and false positive events for alignment or structural variant detection. The figure underscores the advantage of certain example methods in achieving improved sequencing accuracy and efficiency.

The topmost panel A marks the genomic positions of the relevant sequence and highlights the relative location of various genomic elements in subsequent panels. Directly below, the panel B features an annotation track that maps key landmarks such as RefSeq genes, LINE elements, SINE elements, simple repeats, and insertions. This panel B provides the location of these features that are analyzed by various method below. Dashed red lines 602 specifically highlight a repeat section and an inserted region for special attention.

Panel C visualizes the alignment of HiFi assembly reads, represented with a BAM (Binary Alignment Map) file. This portion of the figure displays the quality of the assembly and potential structural variants by showing how well these reads align with the reference genome. Following this, the panel D displays the sequencing coverage depth for the disclosed Rescue assembly, which serves to show the number of times each genomic base is covered by sequencing reads, and is a metric that is useful for assessing assembly quality and high-confidence regions.

Panel D focuses on the coverage of a method of rescue assembly and rescued read depth. Notably, this panel reveals read depth in the inserted region, a typically challenging area for sequencing. This read depth indicates that the disclosed method has been effective in capturing this complex, inserted region, potentially unveiling structural variants that might otherwise be overlooked.

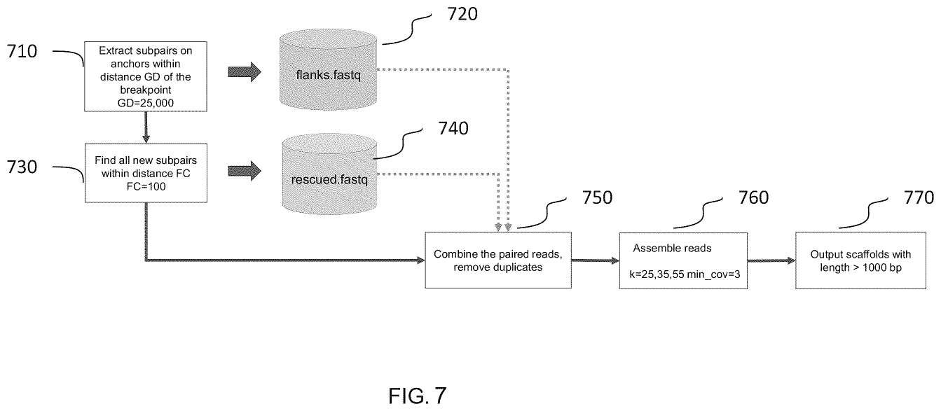

FIG. 7 illustrates an example pipeline designed for structural variant detection in genomic data. The workflow delineates a series of steps of the process from raw sequencing reads to the final assembled scaffolds, which successfully determines the sequence of regions with potential structural variants. The pipeline may be performed on a system designed for genomic analysis, specifically to detect structural variants (SVs) using sequencing data. The system may also include modules for sequencing, read mapping, SV detection, and data output. This pipeline may be implemented through software that processes sequencing data, identifies patterns indicative of SVs, and records these findings. The following steps include examples of file names, for illustration purposes only. Modules according to the following steps may be stored in computer memory and executed by a processor. The first step 710 in the pipeline is “Extract Subpairs on Anchors within Distance GD of the Breakpoint,” where genomic distance is set to, for example, 25,000 base pairs. In some embodiments, the genomic distance may be any of 1 bp, 5 bp, 10 bp, 20 bp, 50 bp, 100 bp, 200 bp, 500 bp, 1 kbp, 10kbp, 20 kbp, 30kbp, 40kbp, 50 kbp, and 100kbp. In this phase, subpairs of reads that are anchored at a distance less than or equal to the genomic distance from the potential breakpoint are extracted. These chosen subpairs are then saved in memory into a file 720, which in the figure is designated as “flanks.fastq.”

Following this, the pipeline advances to the second step, 730, “Find All New Subpairs within a Distance FC,” where FC is flow cell distance that may be measured in arbitrary units, such as unite related to the read ID in the FASTQ file defined in this non-limiting example as 100 units. In some embodiments, the FC may be any of 10, 50, 100, 200, 500, 1,000, 2,000 units. During this step, additional subpairs that lie within the distance FC from the anchors are identified. These are also known as ‘rescued’ subpairs, which are then saved in memory to another file 740 called “rescued.fastq.” This step of saving additional rescued subpairs enhances the robustness of structural variant detection by incorporating additional data that might have been overlooked in initial analyses. By considering spatially correlated reads and rescuing subpairs, the system can used the files saved in memory to run additional methods to potentially identify more complex or subtle structural variants that would otherwise be missed.

The third step 750 in the workflow involves combining the “flanks.fastq” and “rescued.fastq” files. Here, any duplicate paired reads may be identified and removed to ensure any data used in the process is not duplicative as it is processed further. In some embodiments, these steps may improve the memory requirements of the system by limiting the amount of duplicative files and/or by selecting a subset of files to perform analysis upon. Additionally, in some embodiments, the methods will improve the speed and efficiency of assembly processes by narrowly targeting the reads that need to be analyzed to execute the alignment or assembly process.

Upon combining the reads, the next step 760 is the assembly of these reads. This is often accomplished using assembly processes, with SPAdes being used as an example in the figure. As described above with respect to FIG. 2 and FIG. 6, the assembly process transforms the unique subpairs of reads into contiguous sequences, or contigs, which are the building blocks for identifying structural variants. The last shown step 770 of the pipeline focuses on outputting scaffolds that have a length greater than 1,000 base pairs (BP). These scaffolds serve as the assembled genomic regions that are likely to contain structural variants and are thus the primary output of the pipeline for further analysis.

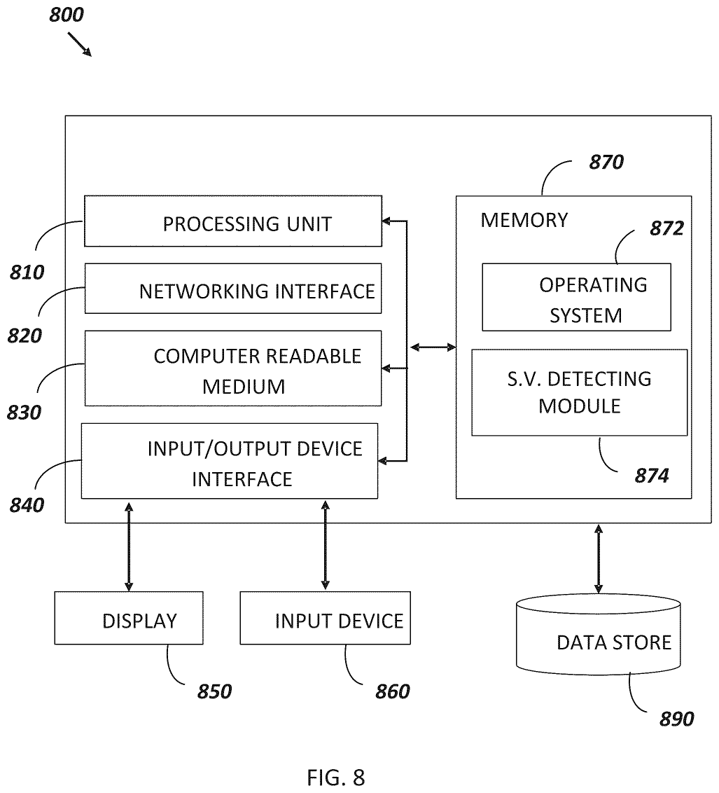

Embodiments of the present disclosure also include a system for analyzing and assembling sequences of polynucleotides. FIG. 8 is a block diagram of an exemplary computing system 800 that may be used in connection with an illustrative sequencing system. The computing system 800 may be configured to determine a DNA sequence by using the sequencing and assembly methods disclosed herein. The general architecture of the computing system 800 includes an arrangement of computer hardware and software components. The computing system 800 may include many more (or fewer) elements. It is not necessary, however, that all of these generally conventional elements be shown in order to provide an enabling disclosure.

As illustrated, the computing system 800 includes a processing unit 810, a network interface 820, a computer-readable medium drive 830, an input/output device interface 840, a display 850, and an input device 860, all of which may communicate with one another by way of a communication bus. The network interface 870 may provide connectivity to one or more networks or computing systems. The processing unit 810 may thus receive information and instructions from other computing systems or services via a network. The processing unit 810 may also communicate to and from memory 870 and further provide output information for an optional display 850 via the input/output device interface 840. The input/output device interface 840 may also accept input from the optional input device 860, such as a keyboard, mouse, digital pen, microphone, touch screen, gesture recognition system, voice recognition system, gamepad, accelerometer, gyroscope, or other input device.

The memory 870 may contain computer program instructions (grouped as modules or components in some embodiments) that the processing unit 810 executes in order to implement one or more embodiments. The memory 870 generally includes RAM, ROM and/or other persistent, auxiliary or non-transitory computer-readable media. The memory 870 may store an operating system 872 that provides computer program instructions for use by the processing unit 810 in the general administration and operation of the computing device 800. The memory 870 may further include computer program instructions and other information for implementing aspects of the present disclosure.

For example, in one embodiment, the memory 870 includes a structural variant detecting module 874 for analyzing and assembling sequences of polynucleotides. The module 874 can perform the methods disclosed herein, including the method described with respect to the flow diagrams of, for example, FIG. 2. In addition, memory 870 may include or communicate with the data store 890 and/or one or more other data stores that store one or more inputs, one or more outputs, and/or one or more results (including intermediate results) of determining a DNA sequence and providing an assembly process according to the present disclosure.

### Systems and Instruments

An aspect of the disclosure is directed to methods for identifying links across an entire genome. In some embodiments, a method may include receiving a BAM file that includes spatial information. The method may proceed by splitting the BAM file into surface and chromosomes. For each subset of the BAM file (surface/chromosome), a “KD-tree” may be constructed, which is a data structure for querying m-dimensional ranges, where m>1. Then, the method may proceed for each point p in each KD-tree t. The KD-tree t may in turn be queried for all points p_neighbors within spatial distance threshold of p. The KD-tree t may be queried for each p2 in p_neighbors. The method may determine a link if p and p2 are within a genomic distance threshold, and then record (p,p2) as a link.

In some embodiments, a method of finding links between read pairs on a flowcell may include the step of providing sequencing data for read pairs from clusters on the flowcell. The method may also include filtering clusters that are spatially distant from one another, and/or filtering clusters that are genomically distant from one another. The method may include selecting neighboring clusters that are within a spatial distance threshold as neighboring clusters; and the assigning links to two read pairs in the neighboring clusters when the clusters are within a genomic distance threshold. In some embodiments, assigning links to two read pairs may occur when the genomic distance threshold is a preset threshold. In some embodiments, the method may generate a first subset of the sequencing data for read pairs by selecting the clusters that are spatially distant from one another. In some embodiments, the method may include selecting a first cluster that has nucleic acid derived from a first chromosome and a second cluster has nucleic acid from a second, different chromosome.

In some embodiments, the clusters are on the same surface but opposite ends of the flowcell. In some embodiments, the clusters are on opposite surfaces of the flowcell. In some embodiments, the calculating the spatial null distribution of the plurality of the plurality of read pairs comprises determining read pairs from clusters on the flowcell. In some embodiments, a first cluster has nucleic acid derived from a first chromosome and a second cluster has nucleic acid from a second, different chromosome.

Various embodiments of the present disclosure may be a system, a method, and/or a computer program product at any possible technical detail level of integration. The computer program product may include a computer readable storage medium (or mediums) having computer readable program instructions thereon for causing a processor to carry out aspects of the present disclosure.

For example, the functionality described herein may be performed as software instructions are executed by, and/or in response to software instructions being executed by, one or more hardware processors and/or any other suitable computing devices. The software instructions and/or other executable code may be read from a computer readable storage medium (or mediums). Computer readable storage mediums may also be referred to herein as computer readable storage or computer readable storage devices.

The computer readable storage medium can be a tangible device that can retain and store data and/or instructions for use by an instruction execution device. The computer readable storage medium may be, for example, but is not limited to, an electronic storage device (including any volatile and/or non-volatile electronic storage devices), a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a solid state drive, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions (as also referred to herein as, for example, “code,” “instructions,” “module,” “application,” “software application,” and/or the like) for carrying out operations of the present disclosure may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. Computer readable program instructions may be callable from other instructions or from itself, and/or may be invoked in response to detected events or interrupts. Computer readable program instructions configured for execution on computing devices may be provided on a computer readable storage medium, and/or as a digital download (and may be originally stored in a compressed or installable format that requires installation, decompression or decryption prior to execution) that may then be stored on a computer readable storage medium. Such computer readable program instructions may be stored, partially or fully, on a memory device (e.g., a computer readable storage medium) of the executing computing device, for execution by the computing device. The computer readable program instructions may execute entirely on a user's computer (e.g., the executing computing device), partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer-readable storage medium having instructions stored therein comprises an article of manufacture including instructions which implement aspects of the function/act specified in the flowchart(s) and/or block diagram(s) block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks. For example, the instructions may initially be carried on a magnetic disk or solid-state drive of a remote computer. The remote computer may load the instructions and/or modules into its dynamic memory and send the instructions over a telephone, cable, or optical line using a modem. A modem local to a server computing system may receive the data on the telephone/cable/optical line and use a converter device including the appropriate circuitry to place the data on a bus. The bus may carry the data to a memory, from which a processor may retrieve and execute the instructions. The instructions received by the memory may optionally be stored on a storage device (e.g., a solid-state drive) either before or after execution by the computer processor.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a service, module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. In addition, certain blocks may be omitted in some implementations. The methods and processes described herein are also not limited to any particular sequence, and the blocks or states relating thereto can be performed in other sequences that are appropriate.

It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions. For example, any of the processes, methods, processes, elements, blocks, applications, or other functionality (or portions of functionality) described in the preceding sections may be embodied in, and/or fully or partially automated via, electronic hardware such application-specific processors (e.g., application-specific integrated circuits (ASICs)), programmable processors (e.g., field programmable gate arrays (FPGAs)), application-specific circuitry, and/or the like (any of which may also combine custom hard-wired logic, logic circuits, ASICs, FPGAs, etc. with custom programming/execution of software instructions to accomplish the techniques).

Any of the above-mentioned processors, and/or devices incorporating any of the above-mentioned processors, may be referred to herein as, for example, “computers,” “computer devices,” “computing devices,” “hardware computing devices,” “hardware processors,” “processing units,” and/or the like. Computing devices of the above-embodiments may generally (but not necessarily) be controlled and/or coordinated by operating system software, such as Mac OS, IOS, Android, Chrome OS, Windows OS (e.g., Windows XP, Windows Vista, Windows 7, Windows 8, Windows 10, Windows 11, Windows Server, etc.), Windows CE, Unix, Linux, SunOS, Solaris, Blackberry OS, VxWorks, or other suitable operating systems. In other embodiments, the computing devices may be controlled by a proprietary operating system. Conventional operating systems control and schedule computer processes for execution, perform memory management, provide file system, networking, I/O services, and provide a user interface functionality, such as a graphical user interface (“GUI”), among other things.

Reference throughout the specification to “one example”, “another example”, “an example”, and so forth, means that a particular element (e.g., feature, structure, and/or characteristic) described in connection with the example is included in at least one example described herein, and may or may not be present in other examples. In addition, it is to be understood that the described elements for any example may be combined in any suitable manner in the various examples unless the context clearly dictates otherwise.

It is to be understood that the ranges provided herein include the stated range and any value or sub-range within the stated range, as if such value or sub-range were explicitly recited. For example, a range from about 2 kbp to about 20 kbp should be interpreted to include not only the explicitly recited limits of from about 2 kbp to about 20 kbp, but also to include individual values, such as about 3.5 kbp, about 8 kbp, about 18.2 kbp, etc., and sub-ranges, such as from about 5 kbp to about 10 kbp, etc. Furthermore, when “about” and/or “substantially” are/is utilized to describe a value, this is meant to encompass minor variations (up to +/−10%) from the stated value.

In some embodiments, the methods may be written in any of various suitable programming languages, for example compiled languages such as C, C #, C++, Fortran, and Java. Other programming languages could be script languages, such as Perl, MatLab, SAS, SPSS, Python, Ruby, Pascal, Delphi, R and PHP. In some embodiments, the methods are written in C, C #, C++, Fortran, Java, Perl, R, Java or Python. In some embodiments, the method may be an independent application with data input and data display modules. Alternatively, the method may be a computer software product and may include classes wherein distributed objects comprise applications including computational methods as described herein.

In some embodiments, the methods may be incorporated into pre-existing data analysis software, such as that found on sequencing instruments. Software comprising computer implemented methods as described herein are installed either onto a computer system directly, or are indirectly held on a computer readable medium and loaded as needed onto a computer system. Further, the methods may be located on computers that are remote to where the data is being produced, such as software found on servers and the like that are maintained in another location relative to where the data is being produced, such as that provided by a third party service provider.

An assay instrument, desktop computer, laptop computer, or server which may contain a processor in operational communication with accessible memory comprising instructions for implementation of systems and methods. In some embodiments, a desktop computer or a laptop computer is in operational communication with one or more computer readable storage media or devices and/or outputting devices. An assay instrument, desktop computer and a laptop computer may operate under a number of different computer based operational languages, such as those utilized by Apple based computer systems or PC based computer systems. An assay instrument, desktop and/or laptop computers and/or server system may further provide a computer interface for creating or modifying experimental definitions and/or conditions, viewing data results and monitoring experimental progress. In some embodiments, an outputting device may be a graphic user interface such as a computer monitor or a computer screen, a printer, a hand-held device such as a personal digital assistant (i.e., PDA, Blackberry, iPhone), a tablet computer (for example, iPAD), a hard drive, a server, a memory stick, a flash drive and the like.

A computer readable storage device or medium may be any device such as a server, a mainframe, a supercomputer, a magnetic tape system and the like. In some embodiments, a storage device may be located onsite in a location proximate to the assay instrument, for example adjacent to or in close proximity to, an assay instrument. For example, a storage device may be located in the same room, in the same building, in an adjacent building, on the same floor in a building, on different floors in a building, etc. in relation to the assay instrument. In some embodiments, a storage device may be located off-site, or distal, to the assay instrument. For example, a storage device may be located in a different part of a city, in a different city, in a different state, in a different country, etc. relative to the assay instrument. In embodiments where a storage device is located distal to the assay instrument, communication between the assay instrument and one or more of a desktop, laptop, or server is commonly via Internet connection, either wireless or by a network cable through an access point. In some embodiments, a storage device may be maintained and managed by the individual or entity directly associated with an assay instrument, whereas in other embodiments a storage device may be maintained and managed by a third party, commonly at a distal location to the individual or entity associated with an assay instrument. In embodiments as described herein, an outputting device may be any device for visualizing data.

An assay instrument, desktop, laptop and/or server system may be used itself to store and/or retrieve computer implemented software programs incorporating computer code for performing and implementing computational methods as described herein, data for use in the implementation of the computational methods, and the like. One or more of an assay instrument, desktop, laptop and/or server may comprise one or more computer readable storage media for storing and/or retrieving software programs incorporating computer code for performing and implementing computational methods as described herein, data for use in the implementation of the computational methods, and the like. Computer readable storage media may include, but is not limited to, one or more of a hard drive, a SSD hard drive, a CD-ROM drive, a DVD-ROM drive, a floppy disk, a tape, a flash memory stick or card, and the like. Further, a network including the Internet may be the computer readable storage media. In some embodiments, computer readable storage media refers to computational resource storage accessible by a computer network via the Internet or a company network offered by a service provider rather than, for example, from a local desktop or laptop computer at a distal location to the assay instrument.

In some embodiments, computer readable storage media for storing and/or retrieving computer implemented software programs incorporating computer code for performing and implementing computational methods as described herein, data for use in the implementation of the computational methods, and the like, is operated and maintained by a service provider in operational communication with an assay instrument, desktop, laptop and/or server system via an Internet connection or network connection.

In some embodiments, a hardware platform for providing a computational environment comprises a processor (i.e., CPU) wherein processor time and memory layout such as random access memory (i.e., RAM) are systems considerations. For example, smaller computer systems offer inexpensive, fast processors and large memory and storage capabilities. In some embodiments, graphics processing units (GPUs) can be used. In some embodiments, hardware platforms for performing computational methods as described herein comprise one or more computer systems with one or more processors. In some embodiments, smaller computer are clustered together to yield a supercomputer network.

In some embodiments, computational methods as described herein are carried out on a collection of inter- or intra-connected computer systems (i.e., grid technology) which may run a variety of operating systems in a coordinated manner. For example, the CONDOR framework (University of Wisconsin-Madison) and systems available through United Devices are exemplary of the coordination of multiple stand-alone computer systems for the purpose dealing with large amounts of data. These systems may offer Perl interfaces to submit, monitor and manage large sequence analysis jobs on a cluster in serial or parallel configurations. One aspect of the disclosure is directed to a workflow module that may be integrated into existing workflows. In some embodiments, a workflow module may be a two-channel sequencing module and may be integrated into a NGS sequence analysis platform, for example the DRAGEN™ Bio-ID platform from Illumina.

### Definitions

The section headings used herein are for organizational purposes only and are not to be construed as limiting the subject matter described.

Unless defined otherwise, all technical and scientific terms used herein have the same meaning as is commonly understood by one of ordinary skill in the art. The use of the term “including” as well as other forms, such as “include”, “includes,” and “included,” is not limiting. The use of the term “having” as well as other forms, such as “have”, “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

The terms “polynucleotide,” “oligonucleotide,” “nucleic acid” and “nucleic acid molecules” are used interchangeably herein and refer to a covalently linked sequence of nucleotides of any length (i.e., ribonucleotides for RNA, deoxyribonucleotides for DNA, analogs thereof, or mixtures thereof) in which the 3′ position of the pentose of one nucleotide is joined by a phosphodiester group to the 5′ position of the pentose of the next. The terms should be understood to include, as equivalents, analogs of either DNA, RNA, cDNA, or antibody-oligo conjugates made from nucleotide analogs and to be applicable to single stranded (such as sense or antisense) and double stranded polynucleotides. The term as used herein also encompasses cDNA, which is complementary or copy DNA produced from an RNA template, for example by the action of reverse transcriptase. This term refers only to the primary structure of the molecule. Thus, the term includes, without limitation, triple-, double- and single-stranded deoxyribonucleic acid (“DNA”), as well as triple-, double- and single-stranded ribonucleic acid (“RNA”). The nucleotides include sequences of any form of nucleic acid. As apparent from the examples below and elsewhere herein, a nucleic acid can have a naturally occurring nucleic acid structure or a non-naturally occurring nucleic acid analog structure. A nucleic acid can contain phosphodiester bonds; however, in some embodiments, nucleic acids may have other types of backbones, comprising, for example, phosphoramide, phosphorothioate, phosphorodithioate, O-methylphosphoroamidite and peptide nucleic acid backbones and linkages. Nucleic acids can have positive backbones; non-ionic backbones, and non-ribose based backbones. Nucleic acids may also contain one or more carbocyclic sugars. The nucleic acids used in methods or compositions herein may be single stranded or, alternatively double stranded, as specified. In some embodiments a nucleic acid can contain portions of both double stranded and single stranded sequence, for example, as demonstrated by forked adapters. A nucleic acid can contain any combination of deoxyribo- and ribonucleotides, and any combination of bases, including uracil, adenine, thymine, cytosine, guanine, inosine, xanthanine, hypoxanthanine, isocytosine, isoguanine, and base analogs such as nitropyrrole (including 3-nitropyrrole) and nitroindole (including 5-nitroindole), etc. In some embodiments, a nucleic acid can include at least one promiscuous base. A promiscuous base can base-pair with more than one different type of base and can be useful, for example, when included in oligonucleotide primers or inserts that are used for random hybridization in complex nucleic acid samples such as genomic DNA samples. An example of a promiscuous base includes inosine that may pair with adenine, thymine, or cytosine. Other examples include hypoxanthine, 5-nitroindole, acylic 5-nitroindole, 4-nitropyrazole, 4-nitroimidazole and 3-nitropyrrole. Promiscuous bases that can base-pair with at least two, three, four or more types of bases can be used.

As used herein, the term “fragment,” when used in reference to a first nucleic acid, is intended to mean a second nucleic acid having a part or portion of the sequence of the first nucleic acid. That is, one or more fragments may be a separable part of an original long strand of polynucleotides. Generally, the fragment and the first nucleic acid are separate molecules. The fragment can be derived, for example, by physical removal from the larger nucleic acid, by replication or amplification of a region of the larger nucleic acid, by degradation of other portions of the larger nucleic acid, a combination thereof or the like. The term can be used analogously to describe sequence data or other representations of nucleic acids.

As used herein, the term “haplotype” refers to a set of alleles at more than one locus inherited by an individual from one of its parents. A haplotype can include two or more loci from all or part of a chromosome. Alleles include, for example, single nucleotide polymorphisms (SNPs), short tandem repeats (STRs), gene sequences, chromosomal insertions, chromosomal deletions etc. The term “phased alleles” refers to the distribution of the particular alleles from a particular chromosome, or portion thereof. Accordingly, the “phase” of two alleles can refer to a characterization or representation of the relative location of two or more alleles on one or more chromosomes.

As used herein “Anchor Reads” are reads that can be mapped with high confidence or unambiguously to unique positions in a genome. Anchor reads serve as reliable reference points in the mapping process, providing high-confidence alignments between the sequence reads and the reference genome. These anchor reads are usually characterized by a high degree of similarity to known sequences in the reference genome, often facilitated by processes that assign high-quality alignment scores based on the number of matches, mismatches, gaps, and other criteria. Essentially, these

Detecting structural variants, which include insertions, deletions, inversions, and translocations, often poses challenges because they inherently involve larger, more complex alterations to the genome than single-nucleotide polymorphisms (SNPs) or small indels. The high-confidence anchor reads become particularly crucial in this context. When reads are mapped to a reference genome, some may align perfectly or nearly perfectly, serving as anchor reads, while others may not align well or may align to multiple locations. These less reliably mapped reads may in fact be indicative of structural variants, and their accurate mapping often relies on the context provided by anchor reads.

For example, in the case of a deletion in the sample genome relative to the reference genome, an anchor read may align well at one end but have a ‘dangling’ other end that doesn't align anywhere in proximity. The presence of a high-confidence anchor read can provide the context needed to recognize that the ‘dangling’ end is not a sequencing error or artifact but is likely part of a structural variant. Likewise, for insertions, translocations, or inversions, anchor reads can offer the stable framework within which the unusual or less confidently mapped reads can be understood.

In paired-end sequencing, one read in the pair might serve as the anchor read while the other spans a structural variant. The anchor read assures that the pair exists in a specific region, giving bioinformaticians confidence to explore what the other read in the pair might reveal about structural changes in the genome. Tools specialized in detecting structural variants often use these anchor reads as starting points for ‘walking’ along the genome to find the boundaries of structural variants.

As used herein, the term “active region” or “region of interest” refers to a segment of the genome that is specifically targeted for sequencing or currently being analyzed during a sequencing method step. These regions may be a single region or a window covering multiple sequence reads at a time. When it comes to methods of assembly or structural variant detection, an active region is often the focal point where advanced sequencing techniques are applied to obtain a highly accurate sequence. In the context of structural variant detection, active regions may be scrutinized using specialized techniques that can detect larger-scale genomic alterations, such as inversions, translocations, or large indels. These variants may not be evident with standard sequencing approaches and often require methods like paired-end or long-read sequencing to span the entire region of interest. This is also relevant for assembling a genome from scratch, where active regions may be targeted for individual steps of a sequencing process to be sequenced with a higher coverage depth or with longer reads to ensure that these important parts of the genome are assembled correctly.

As used herein, the term “Anchor Read” refers to reads that can be mapped with high confidence or unambiguously to unique positions in a genome. Anchor reads serve as reliable reference points in the mapping process, providing high-confidence alignments between the sequence reads and the reference genome. These anchor reads are usually characterized by a high degree of similarity to known sequences in the reference genome, often facilitated by methods that assign high-quality alignment scores based on the number of matches, mismatches, gaps, and other criteria.

As used herein, the term “flanking” genomic sequencing refers to stretches of DNA or RNA fragments that are situated at a certain distance from a specific region of interest, such as an anchor read, a gene, a mutation site, or a repetitive element. These regions may be used as reference points and may not necessarily be directly next to the region of interest. The distance between the flanking region and the target can vary widely, from just a few base pairs to several kilobases away, depending on the genome and the method of used to link reads to anchor reads. For example, some methods of the disclosure are able to link reads from several kilobases away, and may be even more sensitive to structural variants that are several kilobases long.

In the context of anchor sequence reads, as described above, flanking regions serve as reference points for alignment but are not required to be immediately adjacent to the sequence of interest. An anchor read may include sequences that are several hundred or even thousands of base pairs away from the flanking regions. These non-adjacent flanking regions are particularly useful when the anchor read includes repetitive sequences that occur frequently in the genome, or in identifying structural variants. By identifying unique flanking sequences at a distance, methods according to the disclosure can still map the anchor read to the correct location on the genome.

The use of distant flanking regions is a useful strategy of the disclosure for use in genomic sequencing to achieve accurate mapping. It allows for the unambiguous alignment of reads that would otherwise be difficult to place due to the presence of repetitive or complex sequences. By considering a range of distances for potential flanking regions, various tools can effectively ‘anchor’ reads to their proper location in the genome, which is useful for reliable genome assembly and the accurate identification of genetic variants.

As used herein, the term “unambiguous mapping,” in the context of genomic sequencing refers to the process of correctly and uniquely assigning a sequenced DNA fragment to a single location in a reference genome. This means that the sequence of the fragment is so distinctive that it matches one and only one region in the reference genome with a high degree of confidence. By way of example, challenges in mapping may arise because genomes often contain repetitive sequences. If a fragment comes from a repetitive region, it may map to multiple locations, leading to ambiguous mapping. Ambiguity in mapping can complicate genetic analyses and may lead to incorrect conclusions. Therefore, the goal is to achieve unambiguous mapping wherever possible, which is more likely with longer reads, longer synthetic reads, long sequences of linked reads, or with fragments that include unique sequences flanking repetitive regions.

As used herein, the term “ambiguous mapping,” or “ambiguously mapping” refers to a scenario when a fragment of DNA or RNA (a sequence of nucleotides) aligns with two or more locations in the target polynucleotide sequence with low confidence and/or a similar level of confidence for the two or more locations. When sequencing a genome, individual fragments are generated and then will usually be matched back to a reference genome to determine their original location. This process is known as mapping. If a read comes from a unique sequence in the genome, the read can be mapped unambiguously. However, if the read is derived from a sequence that is, for example, repeated in the genome, a mapping process may find multiple potential origins for the read. These multiple matching locations make it unclear where the read actually came from, hence the term “ambiguous mapping”.

As used herein, the term “alignment field” refers to a category of data within an alignment record, specifically detailing the relationship between a sequence read and a reference sequence. These alignment records are generally stored in standard formats like the Sequence Alignment/Map (SAM) file, which is widely used for storing sequence alignment data. The SAM format organizes alignment information into several predefined fields, each field representing a specific aspect of the alignment. For instance, fields such as QNAME (query name), FLAG (alignment properties), RNAME (reference sequence name), and POS (position of alignment) are standard components of an alignment record. Additional fields include MAPQ (mapping quality), indicating the confidence in the alignment, and CIGAR (Compact Idiosyncratic Gapped Alignment Report), which succinctly characterizes how the read aligns to the reference, encompassing matches, mismatches, insertions, and deletions.

As described herein, alignment fields are useful for interpreting the alignment's quality and accuracy. These fields contain information such as the precise (or approximate) starting position of the alignment on the reference sequence, the sequence of the read itself, the quality scores for each base in the read, and details about the read's mate in paired-end sequencing. For example, a CIGAR string is useful in identifying mismatches and gaps that may suggest variations between the read and the reference.

As described herein, an alignment field can also indicate an ambiguous alignment if, for example, the MAPQ score is low, which signifies that the read aligns equally well to multiple locations in the reference genome. Another indication of ambiguity can be inferred from the FLAG field, which may denote whether a read is mapped in a proper pair or not. Reads not properly paired often result from one read of a pair mapping confidently to one location while its mate maps to another, or not at all. In cases where the reference genome contains repetitive sequences, a read derived from such a region might map to several locations with similar scores, leading to ambiguous alignment. Ambiguously aligned reads may be flagged and optionally excluded from further analysis.

As used herein, the terms “background region” or “baseline scenario” (particularly when it involves the use of truth data sets), refer to a set of sequence data that has been validated and is used as a comparative standard for assessing the quality of sequencing efforts. The size of the sequence data may vary from a short sequence to a long sequence up to the size of a reference genome. Background regions may be generated for a section of the sequencing data set and used as a comparison for the rest of the same sequencing data set. For example, a portion of the sequencing data may be evaluated for some metric, such as sequence depth, and used to determine if the rest of the sequencing data (or a portion thereof) is abnormal and indicates some genomic variant.

Truth data sets may include sequences with known variants, including single nucleotide polymorphisms (SNPs), insertions, deletions, and other genetic features that have been verified through rigorous testing and are considered highly accurate. These truth sets may be employed as benchmarks to evaluate how well a new sequencing run can identify and replicate known genetic variations. They provide a point of comparison to determine the error rate of the new sequencing process by highlighting discrepancies between the newly sequenced data and the validated sequences.

As used herein, the term “putative” generally refers to “generally considered or reputed to be,” which implies an assumption based on some evidence, but without conclusive proof. In the context of genomics, when referring to “putative structural variants,” or “candidate structural variant” the term suggests that these are structural changes in the genome—such as deletions, duplications, insertions, inversions, or translocations—that have been identified as possible or likely variations from the reference genome, but have not yet been fully validated. Putative structural variants are typically identified through computational analyses of genomic data as described herein. Methods according to the disclosure can predict these variants by analyzing patterns in sequencing data that suggest deviations from the expected alignment to a reference genome. For instance, reads, or sets of linked reads, that span breakpoint junctions of an inversion, or clusters of reads that indicate a duplication, might lead to the identification of putative structural variants. However, these predictions may require further investigation to determine their validity.

As used herein, the term “threshold distance” in the context of identifying structural variants in a polynucleotide refers to a predefined maximum/minimum distance within which sequence reads must fall relative to anchor sequence reads to be considered relevant, such as, for example, relevant as part of the same structural variant event. The use of threshold distances is useful for filtering out less relevant reads when analyzing high-throughput sequencing data to detect genomic rearrangements such as deletions, insertions, duplications, inversions, or translocations.

As described above, anchor sequence reads are those that can be aligned with high confidence to a known location on the reference genome. In the vicinity of these anchor reads, other reads that do not align as straightforwardly may still be informative for variant detection if they are within a certain proximity—a threshold distance. The range of threshold distances can vary depending on the type of structural variant being investigated and the sequencing technology used. For example, for small Indels (Insertions/Deletions), the threshold distance might be quite small, often in the range of a few bases up to 50 bases, as the changes are relatively close to the anchor reads. For larger structural variants, the threshold distance may be set from a few hundred to several thousand bases. The larger the expected variant, the greater the distance that might be considered. When parts of the chromosome have been rearranged significantly, the threshold distance could be very large, spanning tens to hundreds of thousands of bases, as the reads indicating the breakpoints of such events could be far from the anchor points in the linear genome sequence.

These threshold distances may or may not be arbitrary. The thresholds may be determined based on empirical evidence and statistical models that account for the distribution of reads and the expected frequency of sequencing errors or natural genomic variation. By setting appropriate threshold distances, researchers can minimize false positives (incorrectly calling a variant where there is none) and false negatives (failing to detect an actual variant). The threshold distance as disclosed herein is a useful parameter in bioinformatics pipelines for structural variant detection, balancing sensitivity (detecting true variants) and specificity (not calling false variants).

Note that in the context of spatially linked reads, distance may refer to genomic distance or a physical distance in the flowcell. As used in the disclosure, the term distance may refer to both (e.g., a threshold distance is applied to both genomic distance and physical distance) and/or may be understood in the context to refer to one or the other type of distance.

As used herein, genomic distance refers to the number of base pairs between two points on a sequence within a genome. The genomic distance is a linear measurement that considers the sequence length alone, irrespective of a polynucleotide's three-dimensional structure. For example, if one gene starts at position 100,000 and another gene starts at position 200,000 on a chromosome, the genomic distance between them is 100,000 base pairs. As describe herein, in the context of identifying structural variants, a threshold genomic distance may be set to determine how far apart two reads can be to still be considered as potentially related to the same structural variant. If two reads are within this threshold genomic distance, they may be analyzed together to identify potential deletions, insertions, or other variants.

Similarly, the term “physical distance,” refers to the actual space between two fragments of polynucleotide a flowcell. This distance may reflect the way DNA is fragmented on the flowcell. When applying thresholds to physical distances, researchers are often looking at the interaction between DNA segments in a three-dimensional space, such as in chromosome conformation capture experiments (e.g., Hi-C). A threshold for physical distance may be used to determine whether two DNA fragments are close enough to each other in order to have originated from the same original polynucleotide sequence.

Thresholds for both genomic and physical distances are useful for interpreting complex genomic data. For genomic distances, thresholds may be applied as described herein, in sequence alignment and variant calling methods to decide whether reads should be considered together for variant detection. For instance, in paired-end sequencing, if the distance between two reads exceeds the expected genomic distance based on the insert size, this could indicate a potential deletion or insertion.

For physical distances, thresholds are used in analyzing links between fragments of polynucleotides. Here, thresholds can help identify fragments that are spatially collocated (such as, by example, within a physical distance threshold) more or less frequently than expected versus random chance.

As used herein, the phrase “located spatially close” refers to the proximity of objects of fragments relative to each other or within a given space. In a broad sense, it means that the fragments are near each other in terms of physical distance, which can be measured in units, such as nanometers or units of distance on a flowcell. Defining what is considered “close” is context-dependent. Close may be defined by a threshold distance, which sets a cutoff for how near two points should be to be considered spatially close. Close may also refer generally to distance, such as determining how close two fragments are to each other, and not necessarily imply close proximity.

As used herein, the phrase “Spatially linked read pairs” in the context of genomic sequencing refers to pairs of DNA sequence reads that originate from the same polynucleotide sequence, and are expected to be a certain distance apart based on, for example, the size of the fragments. These read pairs are considered ‘linked’ because they would have been physically connected in the genome before the DNA is fragmented during, for example, library preparation for sequencing.

When determining which sequence reads are linked to other reads, such as anchor sequence reads, spatially linked read pairs are very useful. As described above, an anchor sequence read is a read that has been confidently mapped to a specific location on the reference genome. By looking at the spatially linked pair of a read, researchers can infer where the other fragment should map to the genome. If the second read of the pair does not map where expected (based on the known length of the DNA fragment), this may suggest the presence of a structural variant between the two reads.

As used herein, the term “nucleotide sequence” is intended to refer to the order and type of nucleotide monomers in a nucleic acid polymer. A nucleotide sequence is a characteristic of a nucleic acid molecule and can be represented in any of a variety of formats including, for example, a depiction, image, electronic medium, series of symbols, series of numbers, series of letters, series of colors, etc. The information can be represented, for example, at single nucleotide resolution, at higher resolution (e.g., indicating molecular structure for nucleotide subunits) or at lower resolution (e.g. indicating chromosomal regions, such as haplotype blocks). A series of “A,” “T,” “G,” and “C” letters is a well-known sequence representation for DNA that can be correlated, at single nucleotide resolution, with the actual sequence of a DNA molecule. A similar representation is used for RNA except that “T” is replaced with “U” in the series.

As used herein, the term “solid support” refers to a rigid substrate that is insoluble in aqueous liquid. The substrate can be non-porous or porous. The substrate can optionally be capable of taking up a liquid (e.g., due to porosity) but will typically be sufficiently rigid that the substrate does not swell substantially when taking up the liquid and does not contract substantially when the liquid is removed by drying. A nonporous solid support is generally impermeable to liquids or gases. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefins, polyimides etc.), nylon, ceramics, resins, Zeonor, silica or silica-based materials including silicon and modified silicon, carbon, metals, inorganic glasses, optical fiber bundles, and polymers. Particularly useful solid supports for some embodiments are located within a flowcell apparatus. Exemplary flowcells are set forth in further detail below.

As used herein, the term “flowcell” is intended to mean a chamber having a surface across which one or more fluid reagents can be flowed. Generally, a flowcell will have an ingress opening and an egress opening to facilitate flow of fluid. A flowcell can have multiple surfaces. Examples of flowcells and related fluidic systems and detection platforms that can be readily used in the methods of the present disclosure are described, for example, in Bentley et al, Nature 456:53-59 (2008), WO 04/018497; U.S. Pat. No. 7,057,026; WO 91/06678; WO 07/123744; U.S. Pat. Nos. 7,329,492; 7,211,414; 7,315,019; 7,405,281, and US 2008/0108082, each of which is incorporated herein by reference.

In many embodiments, a solid support to which nucleic acids are attached in a method set forth herein will have a continuous or monolithic surface. Thus, fragments can attach at spatially random locations wherein the distance between nearest neighbor fragments (or nearest neighbor clusters derived from the fragments) will be variable. The resulting arrays will have a variable or random spatial pattern of features. Alternatively, a solid support used in a method set forth herein can include an array of features that are present in a repeating pattern. In such embodiments, the features provide the locations to which modified nucleic acid polymers, or fragments thereof, can attach. Particularly useful repeating patterns are hexagonal patterns, rectilinear patterns, grid patterns, patterns having reflective symmetry, patterns having rotational symmetry, or the like. The features to which a modified nucleic acid polymer, or fragment thereof, attach can each have an area that is smaller than about 1 mm², 500 μm², 100 μm², 25 μm², 10 μm², 5 μm², 1 μm², 500 nm², or 100 nm². Alternatively, or additionally, each feature can have an area that is larger than about 100 nm², 250 nm², 500 nm², 1 μm², 2.5 μm², 5 μm², 10 μm², 100 μm², or 500 μm². A cluster or colony of nucleic acids that result from amplification of fragments on an array (whether patterned or spatially random) can similarly have an area that is in a range above or between an upper and lower limit selected from those exemplified above.

For embodiments that include an array of features on a surface, the features can be discrete, being separated by interstitial regions. Alternatively, some or all of the features on a surface can be abutting (i.e., not separated by interstitial regions). Whether the features are discrete or abutting, the average size of the features and/or average distance between the features can vary such that arrays can be high density, medium density or lower density. High density arrays are characterized as having features with average pitch of less than about 15 μm. Medium density arrays have average feature pitch of about 15 to 30 μm, while low density arrays have average feature pitch of greater than 30 μm. An array useful in the invention can have feature pitch of, for example, less than 100 μm, 50 μm, 10 μm, 5 μm, 1 μm or 0.5 μm. Alternatively, or additionally, the feature pitch can be, for example, greater than 0.1 μm, 0.5 μm, 1 μm, 5 μm, 10 μm, 50 μm, or 100 μm.

As used herein, the term “source” is intended to include an origin for a nucleic acid molecule, such as a tissue, cell, organelle, compartment, or organism. The term can be used to identify or distinguish an origin for a particular nucleic acid in a mixture that includes origins for several other nucleic acids. A source can be a particular organism in a metagenomic sample having several different species of organisms. In some embodiments the source will be identified as an individual origin (e.g., an individual cell or organism). Alternatively, the source can be identified as a species that encompasses several individuals of the same type in a sample (e.g., a species of bacteria or other organism in a metagenomic sample having several individual members of the species along with members of other species as well).

As used herein, the term “surface,” when used in reference to a material, is intended to mean an external part or external layer of the material. The surface can be in contact with another material such as a gas, liquid, gel, polymer, organic polymer, second surface of a similar or different material, metal, or coat. The surface, or regions thereof, can be substantially flat. The surface can have surface features such as wells, pits, channels, ridges, raised regions, pegs, posts or the like. The material can be, for example, a solid support, gel, or the like.

As an example, in some embodiments, fragments derived from a long nucleic acid molecule captured at the surface of a flowcell occur in a line across the surface of the flowcell (e.g., if the nucleic acid was stretched out prior to fragmentation or amplification) or in a cloud on the surface. Further, a physical map of the immobilized nucleic acid can then be generated. The physical map thus correlates the physical relationship of clusters after immobilized nucleic acid is amplified. Specifically, the physical map is used to calculate the probability that sequence data obtained from any two clusters are linked, as described in the incorporated materials of WO 2012/025250. Alternatively, or additionally, the physical map can be indicative of the genome of a particular organism in a metagenomic sample. In this latter case the physical map can indicate the order of sequence fragments in the organism's genome; however, the order need not be specified and instead the mere presence of two or more fragments in a common organism (or other source or origin) can be sufficient basis for a physical map that characterizes a mixed sample and one or more organisms therein.

In some embodiments, the physical map is generated by imaging the solid support to establish the location of the immobilized nucleic acid molecules across the surface. In some embodiments, the immobilized nucleic acid is imaged by adding an imaging agent to the solid support and detecting a signal from the imaging agent. In some embodiments, the imaging agent is a detectable label. Suitable detectable labels, include, but are not limited to, protons, haptens, radionuclides, enzymes, fluorescent labels, chemiluminescent labels, and/or chromogenic agents. For example, in some embodiments, the imaging agent is an intercalating dye or non-intercalating DNA binding agent. Any suitable intercalating dye or non-intercalating DNA binding agent as are known in the art can be used, including, but not limited to those set forth in U.S. 2012/0282617, which is incorporated herein by reference.

In certain embodiments, a plurality of modified nucleic acid molecules is flowed onto a flowcell comprising a plurality of nano-channels. As used herein, the term nano-channel refers to a narrow channel into which a long linear nucleic acid molecule is stretched. In some embodiments, no more than 1, 2, 3, 4, 5, 6, 7, 8, 9, 10, 11, 12, 13, 14, 15, 16, 17, 18, 19, 20, 30, 40, 50, 60 70, 80, 90, 100, 200, 300, 400, 500, 600, 700, 800, 900 or no more than 1000 individual long strands of nucleic acid are stretched across each nano-channel. In some embodiments the individual nano-channels are separated by a physical barrier that prevents individual long strands of target nucleic acid from interacting with multiple nano-channels. In some embodiments, the solid support comprises at least 10, 50, 100, 200, 500, 1000, 3000, 5000, 10000, 30000, 50000, 80000 or at least 100000 nano-channels.

As used herein, the term “target,” when used in reference to a nucleic acid polymer, is intended to distinguish the nucleic acid, for example, from other nucleic acids, modified forms of the nucleic acid, fragments of the nucleic acid, and the like. Any of a variety of nucleic acids set forth herein can be identified as target nucleic acids, examples of which include genomic DNA (gDNA), messenger RNA (mRNA), copy or complimentary DNA (cDNA), and derivatives or analogs of these nucleic acids. Additionally, a target region of a genome may refer to a region of the genome currently under analysis. Similarly, a target read may refer to a selected read that is undergoing analysis.

As used herein, the term “transposase” is intended to mean an enzyme that is capable of forming a functional complex with a transposon element-containing composition (e.g., transposons, transposon ends, transposon end compositions) and catalyzing insertion or transposition of the transposon element-containing composition into a target DNA with which it is incubated, for example, in an in vitro transposition reaction. The term can also include integrases from retrotransposons and retroviruses. Transposases, transposomes and transposome complexes are generally known to those of skill in the art, as exemplified by the disclosure of US Pat. App. Pub. No. 2010/0120098, which is incorporated herein by reference. Although many embodiments described herein refer to Tn5 transposase and/or hyperactive Tn5 transposase, it will be appreciated that any transposition system that is capable of inserting a transposon element with sufficient efficiency to tag a target nucleic acid can be used. In particular embodiments, a preferred transposition system is capable of inserting the transposon element in a random or in an almost random manner to tag the target nucleic acid. As used herein, the term “transposome” is intended to mean a transposase enzyme bound to a nucleic acid. Typically the nucleic acid is double stranded. For example, the complex can be the product of incubating a transposase enzyme with double-stranded transposon DNA under conditions that support non-covalent complex formation. Transposon DNA can include, without limitation, Tn5 DNA, a portion of Tn5 DNA, a transposon element composition, a mixture of transposon element compositions or other nucleic acids capable of interacting with a transposase such as the hyperactive Tn5 transposase.

As used herein, the term “transposon element” is intended to mean a nucleic acid molecule, or portion thereof, that includes the nucleotide sequences that form a transposome with a transposase or integrase enzyme. typically, the nucleic acid molecule is a double stranded DNA molecule. In some embodiments, a transposon element is capable of forming a functional complex with the transposase in a transposition reaction. As non-limiting examples, transposon elements can include the 19-bp outer end (“OE”) transposon end, inner end (“IE”) transposon end, or “mosaic end” (“ME”) transposon end recognized by a wild-type or mutant Tn5 transposase, or the R1 and R2 transposon end as set forth in the disclosure of US Pat. App. Pub. No. 2010/0120098, which is incorporated herein by reference. Transposon elements can comprise any nucleic acid or nucleic acid analogue suitable for forming a functional complex with the transposase or integrase enzyme in an in vitro transposition reaction. For example, the transposon end can comprise DNA, RNA, modified bases, non-natural bases, modified backbone, and can comprise nicks in one or both strands.

A standard NGS sequencing run yields millions of short sequences that are eventually mapped on a reference genome. A percentage of good-quality reads (1-5%) are discarded because of ambiguous genomic location. Increasing read length (2×500 or long-read sequencing), designing a specialized process to map reads on specific regions of the genome (targeted callers), using expensive and time-consuming library preparation, or a combination thereof may be implemented to address the need for disambiguating such reads that would normally be discarded. However, such approaches are costly, laborious, and time intensive. Spatial information (X and Y coordinates) obtained from a solid support surface) can be leveraged to identify fragments that are generated from a single long input fragment and subsequentially be used to improve mapping reads in ambiguous positions.

Various embodiments of the present disclosure may be a system, a method, and/or a computer program product at any possible technical detail level of integration. The computer program product may include a computer readable storage medium (or mediums) having computer readable program instructions thereon for causing a processor to carry out aspects of the present disclosure.

For example, the functionality described herein may be performed as software instructions are executed by, and/or in response to software instructions being executed by, one or more hardware processors and/or any other suitable computing devices. The software instructions and/or other executable code may be read from a computer readable storage medium (or mediums). Computer readable storage mediums may also be referred to herein as computer readable storage or computer readable storage devices.

The computer readable storage medium can be a tangible device that can retain and store data and/or instructions for use by an instruction execution device. The computer readable storage medium may be, for example, but is not limited to, an electronic storage device (including any volatile and/or non-volatile electronic storage devices), a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a solid state drive, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions (as also referred to herein as, for example, “code,” “instructions,” “module,” “application,” “software application,” and/or the like) for carrying out operations of the present disclosure may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. Computer readable program instructions may be callable from other instructions or from itself, and/or may be invoked in response to detected events or interrupts. Computer readable program instructions configured for execution on computing devices may be provided on a computer readable storage medium, and/or as a digital download (and may be originally stored in a compressed or installable format that requires installation, decompression or decryption prior to execution) that may then be stored on a computer readable storage medium. Such computer readable program instructions may be stored, partially or fully, on a memory device (e.g., a computer readable storage medium) of the executing computing device, for execution by the computing device. The computer readable program instructions may execute entirely on a user's computer (e.g., the executing computing device), partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart(s) and/or block diagram(s) block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks. For example, the instructions may initially be carried on a magnetic disk or solid-state drive of a remote computer. The remote computer may load the instructions and/or modules into its dynamic memory and send the instructions over a telephone, cable, or optical line using a modem. A modem local to a server computing system may receive the data on the telephone/cable/optical line and use a converter device including the appropriate circuitry to place the data on a bus. The bus may carry the data to a memory, from which a processor may retrieve and execute the instructions. The instructions received by the memory may optionally be stored on a storage device (e.g., a solid-state drive) either before or after execution by the computer processor.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a service, module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. In addition, certain blocks may be omitted in some implementations. The methods and processes described herein are also not limited to any particular sequence, and the blocks or states relating thereto can be performed in other sequences that are appropriate.

It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions. For example, any of the processes, methods, processes, elements, blocks, applications, or other functionality (or portions of functionality) described in the preceding sections may be embodied in, and/or fully or partially automated via, electronic hardware such application-specific processors (e.g., application-specific integrated circuits (ASICs)), programmable processors (e.g., field programmable gate arrays (FPGAs)), application-specific circuitry, and/or the like (any of which may also combine custom hard-wired logic, logic circuits, ASICs, FPGAs, etc. with custom programming/execution of software instructions to accomplish the techniques).

Any of the above-mentioned processors, and/or devices incorporating any of the above-mentioned processors, may be referred to herein as, for example, “computers,” “computer devices,” “computing devices,” “hardware computing devices,” “hardware processors,” “processing units,” and/or the like. Computing devices of the above-embodiments may generally (but not necessarily) be controlled and/or coordinated by operating system software, such as Mac OS, IOS, Android, Chrome OS, Windows OS (e.g., Windows XP, Windows Vista, Windows 7, Windows 8, Windows 10, Windows 11, Windows Server, etc.), Windows CE, Unix, Linux, SunOS, Solaris, Blackberry OS, VxWorks, or other suitable operating systems. In other embodiments, the computing devices may be controlled by a proprietary operating system. Conventional operating systems control and schedule computer processes for execution, perform memory management, provide file system, networking, I/O services, and provide a user interface functionality, such as a graphical user interface (“GUI”), among other things.

Reference throughout the specification to “one example”, “another example”, “an example”, and so forth, means that a particular element (e.g., feature, structure, and/or characteristic) described in connection with the example is included in at least one example described herein, and may or may not be present in other examples. In addition, it is to be understood that the described elements for any example may be combined in any suitable manner in the various examples unless the context clearly dictates otherwise.

It is to be understood that the ranges provided herein include the stated range and any value or sub-range within the stated range, as if such value or sub-range were explicitly recited. For example, a range from about 2 kbp to about 20 kbp should be interpreted to include not only the explicitly recited limits of from about 2 kbp to about 20 kbp, but also to include individual values, such as about 3.5 kbp, about 8 kbp, about 18.2 kbp, etc., and sub-ranges, such as from about 5 kbp to about 10 kbp, etc. Furthermore, when “about” and/or “substantially” are/is utilized to describe a value, this is meant to encompass minor variations (up to +/−10%) from the stated value.

While several examples have been described in detail, it is to be understood that the disclosed examples may be modified. Therefore, the foregoing description is to be considered non-limiting.

While certain examples have been described, these examples have been presented by way of example only, and are not intended to limit the scope of the disclosure. Indeed, the novel methods described herein may be embodied in a variety of other forms. Furthermore, various omissions, substitutions and changes in the methods described herein may be made without departing from the spirit of the disclosure. The accompanying claims and their equivalents are intended to cover such forms or modifications as would fall within the scope and spirit of the disclosure.

Features, materials, characteristics, or groups described in conjunction with a particular aspect, or example are to be understood to be applicable to any other aspect or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing examples. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable sub-combination. Moreover, although features may be described above as acting in certain combinations, one or more features from a claimed combination can, in some cases, be excised from the combination, and the combination may be claimed as a sub-combination or variation of a sub-combination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some examples, the actual steps taken in the processes illustrated and/or disclosed may differ from those shown in the figures. Depending on the example, certain of the steps described above may be removed or others may be added. Furthermore, the features and attributes of the specific examples disclosed above may be combined in different ways to form additional examples, all of which fall within the scope of the present disclosure.

For purposes of this disclosure, certain aspects, advantages, and novel features are described herein. Not necessarily all such advantages may be achieved in accordance with any particular example. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain examples include, while other examples do not include, certain features, elements, and/or steps. Thus, such conditional language is not generally intended to imply that features, elements, and/or steps are in any way required for one or more examples or that one or more examples necessarily include logic for deciding, with or without user input or prompting, whether these features, elements, and/or steps are included or are to be performed in any particular example.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain examples require the presence of at least one of X, at least one of Y, and at least one of Z.

Language of degree used herein, such as the terms “approximately,” “about,” “generally,” and “substantially” represent a value, amount, or characteristic close to the stated value, amount, or characteristic that still performs a desired function or achieves a desired result.

The scope of the present disclosure is not intended to be limited by the specific disclosures of preferred examples in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

What is claimed is:

1. A system for identifying structural variants in a polynucleotide, comprising:
a memory; and
at least one processor configured to perform a method, comprising:
obtaining sequence reads from a flowcell comprising fragments of a polynucleotide, wherein a probability of the fragments of the polynucleotide being located near each other on the flowcell is correlated with a distance between the fragments of the polynucleotide in the polynucleotide;
determining anchor sequence reads flanking putative structural variants in the polynucleotide; and
identifying structural variants in the polynucleotide by analyzing sequence reads located within a threshold distance to the anchor sequence reads on the flowcell to determine sequence reads linked to the anchor sequence reads.

2. The system of claim 1, wherein the anchor sequence read is a read mapped with a mapping quality (MAPQ) score of at least 20.

3. The system of claim 2, wherein the anchor sequence read is a read mapped with a MAPQ score of at least 30.

4. The system of claim 3, wherein the anchor sequence read is a paired end read that aligns to a reference genome.

5. The system of claim 1, wherein the processor is configured to detect a putative structural variant from variations in read alignments between a reference genome and the sequence reads.

6. The system of claim 5, wherein the variations comprise at least one of a discordant read pair, split read, or soft-clipped read.

7. The system of claim 1, wherein the processor is configured to retrieve unmapped reads that are within a threshold distance to the anchor reads.

8. The system of claim 7, wherein the processor is configured to assemble the retrieved unmapped reads into a contig sequence.

9. The system of claim 8, wherein the processor is configured to assemble the contig sequence by constructing a de Bruijn graph from k-mers of the retrieved reads.

10. The system of claim 6, wherein the processor is configured to align the anchor sequence reads, and retrieve reads, before performing assembly.

11. The system of claim 6, wherein the processor is configured to align the anchor sequence reads, and retrieve reads, after performing assembly.

12. A method for identifying structural variants in a polynucleotide, comprising:
obtaining sequence reads from a flowcell comprising fragments of a polynucleotide, wherein a probability of the fragments of the polynucleotide being located near each other on the flowcell is correlated with a distance between the fragments of the polynucleotide in the polynucleotide;
determining anchor sequence reads flanking putative structural variants in the polynucleotide; and
identifying structural variants in the polynucleotide by analyzing sequence reads located within a threshold distance to the anchor sequence reads on the flowcell to determine sequence reads linked to the anchor sequence reads.

13. The method of claim 12, wherein the anchor sequence read is a read mapped with a mapping quality (MAPQ) score of at least 20.

14. The method of claim 13, wherein the anchor sequence read is a read mapped with a MAPQ score of at least 30.

15. The method of claim 14, wherein the anchor sequence read is a paired end read that aligns to a reference genome.

16. The method of claim 12, further comprising detecting a putative structural variant from variations in read alignments between a reference genome and the sequence reads.

17. The method of claim 16, wherein the variations comprise at least one of a discordant read pair, split read, or soft-clipped read.

18. The method of claim 12, further comprising retrieving unmapped reads that are within a threshold distance to the anchor reads.

19. The method of claim 18, further comprising assembling the retrieved unmapped reads into a contig sequence.

20. The method of claim 19, wherein assembling a contig sequence comprises constructing a de Bruijn graph from k-mers of the retrieved reads.

21. The method of claim 12, wherein retrieving reads further comprises excluding reads aligning to other loci of the genome with high confidence.

22. The method of claim 12, wherein retrieving reads further comprises excluding k-mers that occur below a predetermined frequency.

23. The method of claim 12, wherein retrieving reads further comprises excluding k-mers that correspond to known interspersed retrotransposable elements.

24. The method of claim 12, wherein retrieving reads further comprises realigning reads to a partially assembled contig sequence and excluding reads that do not map to the partially assembled contig sequence.

25. The method of claim 24, wherein the false positive rate of detecting structural variants is reduced relative to an assembled contig sequence that does not exclude reads that do not map to the partially assembled contig sequence.
